(19) United States
(12) Patent Application Publication
(10) Pub. No.: US 2011/0077418 A1
(43) Pub. Date: Mar. 31, 2011

(54) PROCESS FOR THE HYDROGENATION OF IMINES

(75) Inventors: Steen Saaby, Lemvig (DK); Ib Winckelmann, Bovlingbjerg (DK); Kaare Sondergaard, Holstebro (DK); Xianmiao Liang, Liaoning Province (CN); Yanxiong Ke, Shanghai (CN); Xinliang Wang, Shanghai (CN); Jinxing Ye, Shanghai (CN)

(73) Assignee: CHEMINOVA A/S, Lemvig (DK)

(21) Appl. No.: 12/810,088

(22) PCT Filed: Sep. 25, 2009
(86) PCT No.: PCT/CN2009/001081

§ 371 Date: Jun. 22, 2010

(51) Int. Cl. C07F 17/02; C07C 209/70

(52) U.S. Cl. 556/20; 564/415; 556/144

(57) ABSTRACT

A process is provided for the hydrogenation of imines with hydrogen under elevated pressure in the presence of iridium complexes as catalysts and one or more co-catalysts selected among compounds comprising a carbon-halogen bond. Further provided are novel ligands and metal complexes thereof useful for the catalytic hydrogenation of imines with hydrogen.

The novel ligands are compounds of the formula (VII) or formula (VIII) in the form of racemates, mixtures of stereoisomers or optically pure stereoisomers

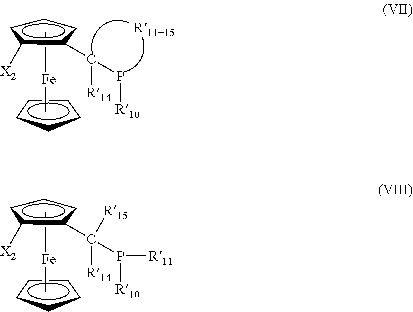

wherein the radicals are as defined in the specification.

### INTRODUCTION

The present invention relates to a process for the hydrogenation of imines with hydrogen under elevated pressure in the presence of iridium complexes as catalysts and one or more co-catalysts in a catalytic effective amount selected among compounds comprising a carbon-halogen bond. The invention further relates to certain novel ligands and metal complexes thereof useful for the catalytic hydrogenation of imines.

### BACKGROUND OF THE INVENTION

Processes for the catalytic hydrogenation of imines have been described in prior art literature. By example, U.S. Pat. No. 4,994,615 describes a process for the asymmetric hydrogenation of prochiral imines wherein homogene iridium based catalysts having chiral diphosphine ligands are used; U.S. Pat. No. 5,112,999 discloses polynuclear iridium compounds and a complex salt of iridium, which contain diphosphine ligands, as catalysts for the hydrogenation of imines; U.S. Pat. No. 6,822,118 describes a process for the asymmetric hydrogenation of prochiral imines with an iridium catalyst in the presence of an ammonium or metal halide and an acid; U.S. Pat. No. 5,859,300 describes a process for the asymmetric hydrogenation of prochiral imines in the presence of an ammonium or metal halide and at least one solid acid with the exception of ion exchangers; U.S. Pat. No. 5,886,225 describes a process for the asymmetric hydrogenation of prochiral imines with an iridium catalyst in the presence of hydroiodic acid (HI) and International patent application no. WO 2007/414769-A1 teaches asymmetric hydrogenation of prochiral imines under elevated pressure in the presence of iridium based catalysts and a phosphonium halide. Among several others, U.S. Pat. No. 5,565,594 disclose iridium or rhodium based catalysts comprising ferrocene diphosphines as ligands for homogeneous catalysis. U.S. Pat. No. 5,244,857; U.S. Pat. No. 5,252,751 U.S. Pat. No. 5,306,853 and U.S. Pat. No. 5,382,729 disclose iridium based catalysts comprising ligands fixed to an inorganic support material such as silicates whereas as International patent application no. WO 97/02232-A1 further suggest that addition of an ammonium or metal halides and at least one acid to the reaction mixtures comprising such heterogeneous iridium based catalysts may improve selectivity and catalyst activity.

Those prior catalysis processes have proved valuable, although it is evident, especially in the case of relatively large batches or on an industrial scale, that the catalysts frequently tend to become deactivated to a greater or lesser extent depending on the catalyst precursor, the substrate and the diphosphine ligands that are used. To prevent the catalyst from being deactivated compounds such as ammonium salts, e.g. ammonium iodide, phosphonium halides or acids have been suggested as additives to the reaction mixture.

### DESCRIPTION OF THE INVENTION

It has now been found, surprisingly, that a comparable catalyst activity can be retained or even increased when the reaction mixture comprises one or more compounds comprising a carbon-halogen bond. Hence, the present invention relates to a process for the hydrogenation of imines with hydrogen under elevated pressure in the presence of iridium based catalysts, optionally in the presence of an inert solvent, wherein the reaction mixture comprises in a catalytic effective amount comprises one or more co-catalysts selected among one or more compounds comprising a carbon-halogen bond whereby the reaction rate and or turn-over number of the iridium based catalyst is increased.

Suitable imines are especially those that contain at least one group

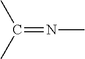

If the groups are substituted asymmetrically and are thus compounds having a prochiral ketimine group, it is possible in the process according to the invention for mixtures of optical isomers or pure optical isomers to be formed if enantioselective or diastereo-selective iridium catalysts are used. The imines may contain further chiral carbon atoms. The free bonds in the above formulae may be saturated with hydrogen or organic radicals having from 1 to 22 carbon atoms or organic hetero radicals having from 1 to 20 carbon atoms and at least one hetero atom from the group O, S, N and P. The nitrogen atom of the group

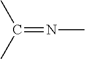

may also be saturated with NH₂ or a primary amino group having from 1 to 22 carbon atoms or a secondary amino group having from 2 to 40 carbon atoms. The organic radicals may be substituted, for example, by F, Cl, Br, C₁-C₄ haloalkyl wherein halogen is preferably F or Cl, —CN, —NO₂, —CO₂H, —CONH₂, —SO₃H, —PO₃H₂, or C₁-C₁₂alkyl esters or amides, or by phenyl esters or benzyl esters of the groups —CO₂H, —SO₃H and —PO₃H₂. Aldimine and ketimine groups are especially reactive, with the result that using the process according to the invention it is possible selectively to hydrogenate

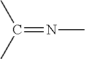

groups in addition to the —C═C— and/or C═O groups. Aldimine and ketimine groups are also to be understood to include

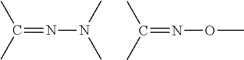

hydrazone and oxime groups.

The process according to the invention is suitable especially for the hydrogenation of aldimines, ketimines and hydrazones with the formation of corresponding amines and hydrazines, respectively. The ketimines are preferably N-substituted. It is preferable to use chiral iridium catalysts and to hydrogenate prochiral ketimines to prepare chiral isomers with optical purity (enantiomeric excess, ee) being, for example, higher than 50%, preferably higher than 70%, and conversions of more than 80% being achievable.

The imines are preferably imines of formula (I)

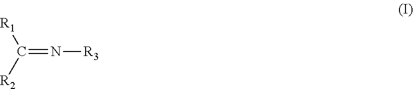

which are hydrogenated to form amines of formula (II)

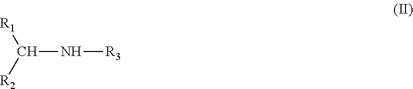

wherein

R₃ is linear or branched C₁-C₁₂alkyl, C₃-C₈cycloalkyl, heterocycloalkyl bonded via a carbon atom and having from 3 to 8 ring atoms and 1 or 2 hetero atoms from the group O, S and NR₆, a C₇-C₁₆aralkyl bonded via an alkyl carbon atom or C₁-C₁₂alkyl substituted by the mentioned cycloalkyl or heterocycloalkyl or heteroaryl;

or wherein

R₃ is C₆-C₁₂aryl, or C₃-C₁₁heteroaryl bonded via a ring carbon atom and having 1, 2 or 3 hetero atoms in the ring; and in either case

the aforementioned R₃ groups being unsubstituted or substituted by one or more substituents, e.g. by —CN, —NO₂, F, Cl, C₁-C₁₂alkoxy, C₁-C₁₂alkylthio, C₁-C₁₂haloalkyl, —OH, C₆-C₁₂aryl or -aryloxy or -arylthio, C₇-C₁₆-aralkyl or -aralkoxy or -aralkylthio, secondary amino having from 2 to 24 carbon atoms, —CONR₄R₅ or by —COOR₄, and the aryl radicals and the aryl groups in the aralkyl, aralkoxy and aralkylthio in turn being unsubstituted or substituted by one or more substituents, e.g. selected among —CN, —NO₂, halogen (e.g. F or Cl), C₁-C₄-alkyl, -alkoxy or -alkylthio, —OH, —CONR₄R₅ or by —COOR₄;

R₄ and R₅ are each independently of the other hydrogen, C₁-C₁₂alkyl, phenyl or benzyl, or R₄ and R₅ together are tetra- or penta-methylene or 3-oxapentylene;

R₁ and R₂ are each independently of the other a hydrogen atom, C₁-C₁₂alkyl or C₃-C₈cycloalkyl, each of which is unsubstituted or substituted independently of the other by one or more substituents, e.g. by —OH, C₁-C₁₂alkoxy, phenoxy, benzyloxy, secondary amino having from 2 to 24 carbon atoms, —CONR₄R₅ or by —COOR₄; or C₆-C₁₂aryl, C₇-C₁₆aralkyl that is unsubstituted or substituted as R₃; or

R₃ is as defined hereinbefore and R₁ and R₂ together represents an alkylene bridge having from 2 to 6 carbon atoms that is optionally interrupted by 1 or 2—O—, —S— or —NR₆— radicals, and/or unsubstituted or substituted by ═O or as R₁ and R₂ above in the meaning of alkyl, and/or condensed with benzene, pyridine, pyrimidine, furan, thiophene or pyrrole; or

R₂ is as defined hereinbefore and R₁ and R₃ together represents an alkylene bridge having from 2 to 6 carbon atoms that is optionally interrupted by 1 or 2—O—, —S— or —NR₆— radicals, and/or unsubstituted or substituted by ═O or as R₁ and R₂ above in the meaning of alkyl, and/or condensed with benzene, pyridine, pyrimidine, furan, thiophene or pyrrole.

R₆ represents hydrogen, C₁-C₁₂alkyl, phenyl or benzyl.

The radicals R₁, R₂ and R₃ may contain one or more chiral centers.

R₁, R₂ and R₃ can be substituted in any positions by identical or different radicals, for example by from 1 to 5, preferably from 1 to 3, substituents.

Suitable substituents for R₁, R₂ and/or R₃ are:

C₁-C₁₂—, preferably C₁-C₆—, and especially C₁-C₄-alkyl, -alkoxy or -alkylthio, e.g. methyl, ethyl, propyl, n-, i- and t-butyl, the isomers of pentyl, hexyl, octyl, nonyl, decyl, undecyl and dodecyl, and corresponding alkoxy and alkylthio radicals; C₁-C₆—, preferably C₁-C₄-haloalkyl having preferably F and Cl as halogen, e.g. trifluoro- or trichloro-methyl, difluorochloromethyl, fluorodichloromethyl, 1,1-difluoroeth-1-yl, 1,1-dichloroeth-1-yl, 1,1,1-trichloro- or 1,1,1-trifluoroeth-2-yl, pentachloroethyl, penta-fluoroethyl, 1,1,1-trifluoro-2,2-dichloroethyl, n-perfluoropropyl, iso-perfluoropropyl, n-perfluorobutyl, fluoro- or chloro-methyl, difluoro- or dichloro-methyl, 1-fluoro- or 1-chloro-eth-2-yl or -eth-1-yl, 1-, 2- or 3-fluoro- or 1-, 2- or 3-chloro-prop-1-yl or -prop-2-yl or -prop-3-yl, 1-fluoro- or 1-chloro-but-1-yl, -but-2-yl, -but-3-yl or -but-4-yl, 2,3-dichloro-prop-1-yl, 1-chloro-2-fluoro-prop-3-yl, 2,3-dichlorobut-1-yl; -aryloxy or -arylthio, in which aryl is preferably naphthyl and especially phenyl, C₇-C₁₆-aralkyl, -aralkoxy and -aralkylthio, in which the aryl radical is preferably naphthyl and especially phenyl and the alkylene radical is linear or branched and contains from 1 to 10, preferably from 1 to 6 and especially from 1 to 3, carbon atoms, for example benzyl, naphthylmethyl, 1- or 2-phenyl-eth-1-yl or -eth-2-yl, 1-, 2- or 3-phenyl-prop-1-yl, -prop-2-yl or -prop-3-yl, with benzyl being especially preferred; the radicals containing the aryl groups mentioned above may in turn be mono- or poly-substituted, for example by C₁-C₄-alkyl, -alkoxy or -alkylthio, halogen, —OH, —CONR₄R₅ or by —COOR₄, R₄ and R₅ are as defined above and examples are methyl, ethyl, n- and iso-propyl, butyl, corresponding alkoxy and alkylthio radicals, F, Cl, Br, dimethyl-, methyl-ethyl- and diethyl-carbamoyl and methoxy-, ethoxy-, phenoxy- and benzyloxy-carbonyl; halogen, preferably F and Cl; secondary amino having from 2 to 24, preferably from 2 to 12 and especially from 2 to 6 carbon atoms, the secondary amino preferably containing 2 alkyl groups, for example dimethyl-, methylethyl-, diethyl-, methylpropyl-, methyl-n-butyl, di-n-propyl-, di-n-butyl-, di-n-hexyl-amino;

—CONR₄R₅, wherein R₄ and R₅ are each independently of the other C₁-C₁₂—, preferably C₁-C₆—, and especially C₁-C₄-alkyl, or R₄ and R₅ together are tetra- or penta-methylene or 3-oxapentylene, the alkyl being linear or branched, e.g. dimethyl-, methylethyl-, diethyl-, methyl-n-propyl-, ethyl-n-propyl-, di-n-propyl-, methyl-n-butyl-, ethyl-n-butyl-, n-propyl-n-butyl- and di-n-butyl-carbamoyl;

—COOR₄, wherein R₄ is C₁-C₁₂, preferably C₁-C₆-alkyl, which may be linear or branched, e.g. methyl, ethyl, n- and i-propyl, n-, i- and t-butyl, and the isomers of pentyl, hexyl, heptyl, octyl, nonyl, decyl, undecyl and dodecyl.

R₁, R₂ and R₃ may contain especially functional groups, such as keto groups, —CN, —NO₂, carbon double bonds, N—O—, aromatic halogen groups and amide groups.

Heteroaryl R₁, R₂ or R₃ are preferably a 5- or 6-membered ring having 1, 2 or 3 identical or different hetero atoms, especially O, S or N, which contains preferably 4 or 5 carbon atoms and can be condensed with benzene. Examples of heteroaromatics from which R₁ or R₂ can be derived are furan, pyrrole, thiophene, pyridine, pyrimidine, indole, quinoline and triazole.

R₁, R₂ or R₃ as heteroaryl-substituted alkyl are derived preferably from a 5- or 6-membered ring having 1 or 2 identical or different hetero atoms, especially O, S or N, which contains preferably 4 or 5 carbon atoms and can be condensed with benzene. Examples of heteroaromatics are furan, pyrrole, thiophene, pyridine, pyrimidine, indole and quinoline.

R₁, R₂ or R₃ as heterocycloalkyl or as heterocycloalkyl-substituted alkyl contain preferably 5 or 6 ring atoms and 1, 2 or 3 identical or different hetero atoms from the group O, S and NR₆, wherein R₆ is as defined above and examples of R₆ include hydrogen, phenyl or benzyl. They can be condensed with benzene. It may be derived, for example, from pyrrolidine, tetrahydrofuran, triazole, tetrahydrothiophene, indane, pyrazolidine, oxazolidine, piperidine, piperazine or morpholine.

Alkyl R₁, R, or R₃ are preferably unsubstituted or substituted by C₁-C₆—, especially by C₁-C₄-alkyl, which may be linear or branched. Examples are -methyl, ethyl, i- and n-propyl, i-, n- and t-butyl, the isomers of pentyl, hexyl, heptyl, octyl, nonyl, decyl, undecyl and dodecyl.

Unsubstituted or substituted cycloalkyl R₁, R₂ or R₃ contain preferably from 3 to 6, especially 5 or 6, ring carbon atoms. Examples are cyclopropyl, cyclobutyl, cyclopentyl, cyclohexyl, cycloheptyl and cyclooctyl.

R₁, R₂ or R₃ as aryl are preferably unsubstituted or substituted naphthyl and especially phenyl.

R₁, R₂ or R₃ as aralkyl are preferably unsubstituted or substituted phenylalkyl having from 1 to 10, preferably from 1 to 6 and especially from 1 to 4 carbon atoms in the alkylene, the alkylene being linear or branched. Examples are especially benzyl, and 1-phenyleth-1-yl, 2-phenyleth-1-yl, 1-phenylprop-1-yl, 1-phenylprop-2-yl, 1-phenyl-prop-3-yl, 2-phenylprop-1-yl, 2-phenylprop-2-yl and 1-phenylbut-4-yl.

In —CONR₄R₅ and —COOR₄, R₄ and R₅ are preferably C₁-C₆—, especially C₁-C₄-alkyl, or R₄ and R₅ together are tetramethylene, pentamethylene or 3-oxapentylene.

Alkylene R₁ and R₂ together or R₁ and R₃ together are preferably interrupted by 1 —O—, —S— or —NR₆—, preferably —O—. R₁ and R₂ together or R₁ and R₃ together form, with the carbon atom or with the —N═C group to which they are bonded, respectively, preferably a 5- or 6-membered ring. For the substituents the preferences mentioned hereinbefore apply. As condensed alkylene, R₁ and R₂ together or R₁ and R₃ together are preferably alkylene condensed with benzene or pyridine. Examples of alkylene are: ethylene, 1,2- or 1,3-propylene, 1,2-, 1,3- or 1,4-butylene, 1,5-pentylene and 1,6-hexylene. Examples of interrupted or ═O— substituted alkylene are 2-oxa-1,3-propylene, 2-oxa-1,4-butylene, 2-oxa- or 3-oxa-1,5-pentylene, 2-methylimino-1,3-propylene, 3-thia-1,5-pentylene, 2-thia-1,4-butylene, 2-thia-1,3-propylene, 2-ethylimino-1,4-butylene, 2- or 3-methyl-imino-1,5-pentylene, 1-oxo-2-oxa-1,3-propylene, 1-oxo-2-oxa-1,4-butylene, 2-oxo-3-oxa-1,4-butylene, 1-oxa-2-oxo-1,5-pentylene.

R₄ and R₅ are preferably each independently of the other hydrogen, C₁-C₄alkyl, phenyl or benzyl.

R₆ is preferably hydrogen or C₁-C₄alkyl.

Another preferred group is formed by prochiral imines in which in formula (I) R₁, R₂ and R₃ are each different from the others and are not hydrogen.

In an especially preferred group, in formula (I) R₃ is 2,6-di-C₁-C₄alkylphen-1-yl or 2,4-di-C₁-C₄alkylthiophen-3-yl and especially 2,6-dimethylphen-1-yl, 2-methyl-6-ethylphen-1-yl or 2,4-dimethylthiophen-3-yl, R₁ is C₁-C₄alkyl and especially ethyl or methyl, and R, is C₁-C₄alkyl, C₁-C₄alkoxymethyl or C₁-C₄alkoxyethyl, and especially methoxymethyl.

Of those compounds, imines of formulae (Ia), (Ib) and (Ic)

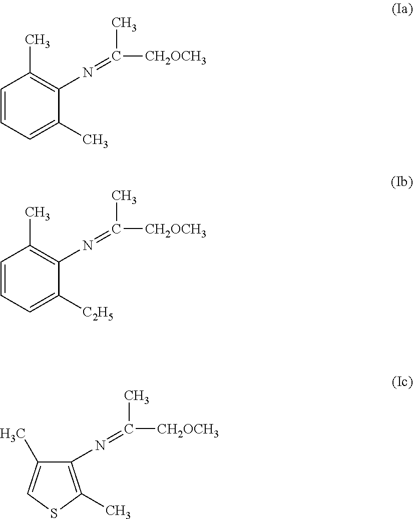

are especially important. Imines of formula (I) are known or they can be prepared in accordance with known processes from aldehydes or ketones and primary amines.

The iridium based catalysts are preferably homogeneous catalysts that are substantially soluble in the reaction medium, but may also be bound to an inorganic support material such as silicates. The term “catalyst” also includes catalyst precursors that are converted into an active catalyst species at the beginning of a hydrogenation. The catalysts may correspond to the formulae (IV), (IVa), (IVb), (IVc) or (IVd):

[XIrYZ] (IV)

[XIrY]⁺A⁻ (IVa)

[YIrZ₄]^{-M}⁺ (IVb)

[YIrHZ₂]₂ (IVc)

[YIrZ₃]₂ (IVd)

[YIrZH(A)] (IVe)

[YIrH(A)₂] (IVf)

[YIr(A)₃] (IVg)

wherein X is two olefin ligands or a diene ligand, Y is a ditertiary diphosphine

(a) the phosphine groups of which are bonded to different carbon atoms of a carbon chain having from 2 to 4 carbon atoms, or

(b) the phosphine groups of which are either bonded directly or via a bridge group —CRₐR_{b}— in the ortho positions of a cyclopentadienyl ring or are each bonded to a cyclopentadienyl ring of a ferrocenyl, or

(c) one phosphine group of which is bonded to a carbon chain having 2 or 3 carbon atoms and the other phosphine group of which is bonded to an oxygen atom or a nitrogen atom bonded terminally to that carbon chain, or

(d) the phosphine groups of which are bonded to the two oxygen atoms or nitrogen atoms bonded terminally to a C₂-carbon chain;

with the result that in the cases of (a), (b), (c) and (d) a 5-, 6-, 7-, 8- or 9-membered ring is formed together with the Ir atom;

the radicals Z are each independently of the other(s) Cl, Br or I;

A is the anion of an oxy or complex acid;

M⁺ is cation such as a phosphonium, a metal or a quaternary ammonium cation; and

Rₐ and R_{b}, are each independently of the other hydrogen, C₁-C₁₂alkyl, C₁-C₄fluoroalkyl, C₃-C₈cycloalkyl, C₆-C₁₂aryl or C₃-C₁₂heteroaryl having heteroatoms selected from the group consisting of O, S and N, which are unsubstituted or substituted independently by the others by one or more substituents, e.g. by C₁-C₆alkyl, C₁-C₆alkoxy, C₃-C₈cycloalkyl, C₃-C₈cycloalkoxy, phenyl, C₁-C₆alkylphenyl, C₁-C₆-alkoxy-phenyl, C₃-C₈heteroaryl or halogen. R_{b} is preferably hydrogen.

The diphosphine Y contains preferably at least one chiral carbon atom and is especially a single stereoisomer (enantiomer or diastereoisomer), or a pair of diastereoisomers. The use of catalysts containing those ligands leads to optical induction in hydrogenation reactions.

X as an olefin ligand may be a branched or, preferably, linear C₂-C₁₂alkylene, especially C₂-C₆alkylene. Some examples are dodecylene, decylene, octylene, 1-, 2- or 3-hexene, 1-, 2- or 3-pentene, 1- or 2-butene, propene and ethene.

X as a diene ligand may be open-chain or cyclic dienes having from 4 to 12, preferably from 5 to 8, carbon atoms, the diene groups preferably being separated by one or two saturated carbon atoms. Some examples are butadiene, pentadiene, hexadiene, heptadiene, octadiene, decadiene, dodecadiene, cyclopentadiene, cyclohexadiene, cycloheptadiene, cyclooctadiene and bridged cyclo-dienes such as norbornadiene and bicyclo-2,2,2-octadiene. Hexadiene, cyclooctadiene (cod) and norbornadiene are preferred.

The phosphine groups contain two identical or different unsubstituted or substituted hydro-carbon radicals having from 1 to 20, especially from 1 to 12 carbon atoms. Preference is given to diphosphines wherein the secondary phosphine groups contain two identical or different radicals from the following group: linear or branched C₁-C₁₂alkyl; unsubstituted or C₁-C₆alkyl- or C₁-C₆alkoxy-substituted C₅-C₁₂-cycloalkyl, C₅-C₁₂cycloalkyl-CH₂—, phenyl or benzyl; and phenyl or benzyl substituted by halogen, C₁-C₆haloalkyl, (C₁-C₁₂alkyl)₃Si, (C₆-C₁₂aryl)₃Si, C₁-C₆haloalkoxy (e.g. trifluoromethoxy), —NH₂, (phenyl)₂N—, (benzyl)₂N—, morpholinyl, piperidinyl, pyrrolidinyl, (C₁-C₁₂alkyl)₂N—, (C₇-C₁₂aralkyl)₂N—, -ammonium-X₁, —SO₃M₁, —CO₂M₁, —PO₃M₁ or by —COO—C₁-C₆-alkyl (e.g. —COOCH₃), wherein M₁ is an alkali metal or hydrogen and X₁ is the anion of a monobasic acid. M₁ is preferably H, Li, Na or K. X₁, as the anion of a monobasic acid, is preferably Cl, Br or the anion of a carboxylic acid, for example formate, acetate, trichloroacetate or trifluoroacetate.

A secondary phosphine group may also be a radical of the formulae

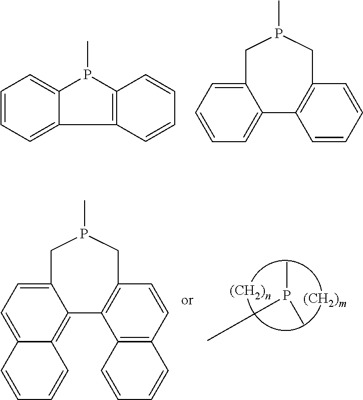

wherein the rings may be substituted by, e.g., C₁-C₆-alkyl, C₁-C₆-alkoxy, C₁-C₆-alkoxy-C₁-C₆-alkyl, phenyl, benzyl, benzyloxy or C₁-C₆-alkylidenedioxyl or C₁-C₆-alkylenedioxyl and wherein m and n are each independently of the other an integer from 1 to 10, and the sum of m+n is from 1 to 12, especially from 4 to 8. Examples thereof are [3.3.1]- and [4.2.1]-phobyl of the formulae

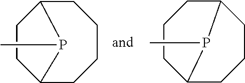

Examples of alkyl that preferably contains from 1 to 6 carbon atoms are methyl, ethyl, n-propyl, isopropyl, n-, i- and t-butyl and the isomers of pentyl and hexyl. Examples of un-substituted or alkyl-substituted cycloalkyl are cyclopentyl, cyclohexyl, methyl- or ethyl-cyclohexyl and dimethylcyclohexyl. Examples of alkyl-, alkoxy- or haloalkoxy-substituted phenyl and benzyl are methylphenyl, dimethylphenyl, trimethylphenyl, ethyl-phenyl, methylbenzyl, methoxyphenyl, dimethoxyphenyl, trifluoromethylphenyl, bis-trifluoromethylphenyl, trifluoromethoxyphenyl and bis-trifluoro-methoxyphenyl. Preferred phosphine groups are those that contain identical or different, preferably identical, radicals from the group C₁-C₆alkyl; cyclopentyl and cyclohexyl that are unsubstituted or have from 1 to 3 C₁-C₄alkyl or C₁-C₄alkoxy substituents, benzyl and, especially, phenyl that is unsubstituted or has from 1 to 3 C₁-C₄alkyl, C₁-C₄alkoxy, F, Cl, C₁-C₄fluoroalkyl or C₁-C₄fluoroalkoxy substituents.

Examples of secondary non-cyclic phosphine groups include —P(C₁-C₆-alkyl)₂, —P(C₅-C₈-cycloalkyl)₂, —P(C₇-C₁₂-bicycloalkyl)₂, —P(o-furyl)₂, —P(C₆H₅)₂, —P[2-(C₁-C₆-alkyl)C₆H₄]₂, —P[3-(C₁-C₆-alkyl)C₆H₄]₂, —P[4-(C₁-C₆-alkyl)C₆H₄]₂, —P[2-C₁-C₆-alkoxy)C₆H₄]₂, —P[3-(C₁-C₆-alkoxy)C₆H₄]₂, —P[4-(C₁-C₆-alkoxy)C₆H₄]₂, —P[2-(trifluoromethyl)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(C₁-C₆-alkyl)₂C₆H₃]₂, —P[3,5-bis(C₁-C₆-alkoxy)₂C₆H₃]₂, —P[3,5-bis(C₁-C₆alkyl)₂-4-(C₁-C₆-alkoxy)C₆H₂]₂ and —P[3,4,5-tris(C₁-C₆-alkoxy)₂C₆H₃]₂.

Depending on the type of substitution and the number of substituents, the cyclic phosphine groups can be C-chiral, P-chiral or C- and P-chiral.

Examples of secondary cyclic phosphine groups can correspond to the following formulae:

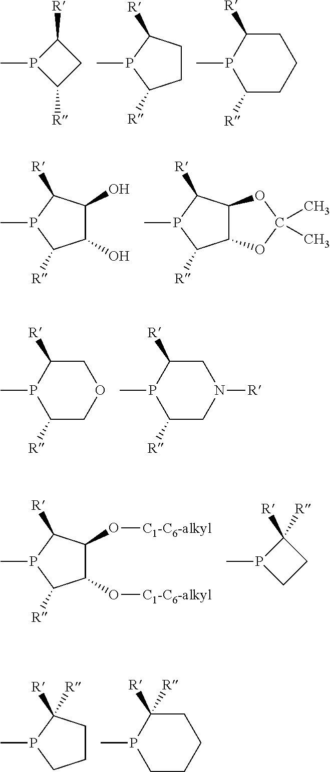

where the substituents R′ and R″ are identical or different and may each represent hydrogen, C₁-C₆alkyl, for example methyl, ethyl, n- or i-propyl, benzyl, or —CH₂—O—C₁-C₆alkyl or —CH₂—O—C₆-C₁₀aryl, and R′ and R″ are identical or different. When R′ and R″ are bound to the same carbon atom, they can together form a C₄-C₆alkylene group. In the above formulae, when applicable e.g. when R′ and R″ are not both H or identical and at the same time attached to the same carbon atom, only one of the possible diastereomers of each formula is indicated.

Y as a diphosphine may be represented by one of formula (V), (Va), (Vb), (Vc), (Vd) or (Ve),

R₇R₈P—R₉—PR₁₀R₁₁ (V),

R₇R₈P—O—R₁₂—PR₁₀R₁₁ (Va),

R₇R₈P—NR_{c} R₁₂—PR₁₀R₁₁ (Vb),

R₇R₈P—O—R₁₃—O—PR₁₀R₁₁ (Vc),

R₇R₈P—NR_{c}—R₁₃—NR_{c}—PR₁₀R₁₁ (Vd),

R₇R₈P—NR_{c}—R₉—PR₁₀R₁₁ (Ve)

wherein R₇, R₈, R₁₀ and R₁₁ each independently of the others represent C₁-C₁₂alkyl, C₁-C₁₂alkoxy, C₃-C₈cycloalkyl, C₃-C₈cycloalkoxy, C₆-C₁₂aryl, C₃-C₁₂heteroaryl, C₆-C₁₂aryl-C₁-C₁₂alkyl-, C₆-C₁₂aryl-C₁-C₁₂alkoxy- or C₃-C₁₂heteroaryl-C₁-C₁₂alkyl- having heteroatoms selected from the group consisting of O, S and N, the aforementioned groups being unsubstituted or substituted independently of the others by one or more substituents e.g. by C₁-C₆alkyl, C₁-C₆alkoxy, C₃-C₈cycloalkyl, C₃-C₈cycloalkoxy, phenyl, phenyl-C₁-C₆alkyl- (e.g. benzyl), phenyl-C₁-C₆alkoxy-, C₃-C₈heteroaryl, (C₁-C₁₂alkyl)₃Si, (C₆-C₁₂aryl)₃Si, —NH₂, (phenyl)₂N—, (benzyl)₂N—, morpholinyl, piperidinyl, pyrrolidinyl, (C₁-C₁₂alkyl)₂N—, (C₇-C₁₂aralkyl)₂N—, C₁-C₁₂haloalkyl, C₁-C₁₂haloalkoxy, halogen, -ammonium-X₁, —SO₃M₁, —CO₂M₁, —PO₃M₁ or by —COO—-C₁-C₆alkyl, wherein M₁ is an alkali metal or hydrogen and X, is the anion of a monobasic acid. M, is preferably H, Li, Na or K. X₁, as the anion of a monobasic acid, is preferably Cl, Br or the anion of a carboxylic acid, for example formate, acetate, trichloroacetate or trifluoroacetate; or R₇ and R₈ together or R₁₀ and R₁₁ together represents an alkylene bridge having from 2 to 6 carbon atoms that is optionally interrupted by 1 or more —O—, —S— or —NR₆— comprising radicals such as N—H, N—C₁-C₁₂alkyl or N—C₆-C₁₂aryl; and may be unsubstituted or substituted by ═O or by one or more groups of C₁-C₆alkyl, C₁-C₆alkoxy, C₃-C₈cycloalkyl, C₃-C₈cycloalkoxy, phenyl, phenyl-C₁-C₆alkyl-, phenyl-C₁-C₆alkoxy-, or halogen groups; and/or condensed with benzene, pyridine, pyrimidine, furan, thiophene or pyrrole;

Preferably R₇, R₈, R₁₀ and R₁₁, are identical or different, most preferably identical, radicals selected from the following group: C₁-C₆alkyl; C₄-C₆cycloalkyl that are unsubstituted or have from 1 to 3 C₁-C₄alkyl or C₁-C₄alkoxy substituents; phenyl or benzyl, especially phenyl, that is unsubstituted or has from 1 to 3 C₁-C₄alkyl, C₁-C₄alkoxy, F, Cl, C₁-C₄fluoroalkyl or C₁-C₄fluoroalkoxy substituents.

R₇, R₈, R₁₀ and R₁₁ can be substituted in any positions by identical or different radicals, for example by from 1 to 5, preferably from 1 to 3, substituents

R₉ is linear C₂-C₄alkylene that is unsubstituted or substituted by C₁-C₆alkyl, C₃-C₆-cycloalkyl, phenyl, naphthyl or by benzyl; 1,2- or 1,3-cycloalkylene or -cycloalkenylene, -bicycloalkylene or -bicycloalkenylene having from 4 to 10 carbon atoms, the aforementioned groups each being unsubstituted or substituted independently of one another by one or more substituents, e.g. by C₁-C₆alkyl, phenyl or by benzyl; 1,2- or 1,3-cycloalkylene or -cycloalkenylene, -bicycloalkylene or -bicycloalkenylene having from 4 to 10 carbon atoms, the aforementioned groups each being unsubstituted or substituted independently of one another by one or more substituents, e.g. by C₁-C₆alkyl, phenyl or by benzyl, and in the 1- and/or 2-positions or in the 3-position of which methyl-ene or C₂-C₄alkylidene is bonded; 1,4-butylene substituted in the 2,3-positions by

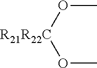

and unsubstituted or substituted in the 1,4-positions by C₁-C₆alkyl, phenyl or by benzyl, wherein R₂₁ and R₂₂ are each independently of the other hydrogen, C₁-C₆alkyl, phenyl or benzyl; 3,4- or 2,4-pyrrolidinylene or 2-methylene-pyrrolidin-4-yl the nitrogen atom of which is substituted by hydrogen, C₁-C₁₂alkyl, phenyl, benzyl, C₁-C₁₂alkoxycarbonyl, C₁-C₈acyl or by or C₁-C₁₂alkylaminocarbonyl; or 1,2-phenylene, 2-benzylene, 1,2-xylylene, 1,8-naphthylene, 2,2′-dinaphthylene or 2,2′-diphenylene, the aforementioned groups each being unsubstituted or substituted independently of one another by one or more substituents. e.g. by C₁-C₄alkyl;

R₁₂ is linear C₂- or C₃-alkylene that is unsubstituted or substituted e.g. by C₁-C₆alkyl, C₃-C₆cycloalkyl, phenyl, naphthyl or by benzyl; 1,2- or 1,3-cycloalkylene or -cycloalkenylene, -bicycloalkylene or -bicycloalkenylene having from 4 to 10 carbon atoms, the aforementioned groups each being unsubstituted or substituted independently of the others by one or more groups, e.g. by C₁-C₆alkyl, phenyl or by benzyl; or 1,2- or 1,3-cycloalkylene or -cycloalkenylene, -bicycloalkylene or -bicycloalkenylene having from 4 to 10 carbon atoms, the aforementioned groups each being unsubstituted or substituted independently of the others by one or more groups, e.g. by C₁-C₆alkyl, phenyl or by benzyl, and in the 1- and/or 2-positions or in the 3-position of which methylene or C₂-C₄alkylidene is bonded; 3,4- or 2,4-pyrrolidinylene or 3-methylene-pyrrolidin-4-yl the nitrogen atom of which is substituted by hydrogen, C₁-C₁₂alkyl, phenyl, benzyl, C₁-C₁₂alkoxycarbonyl, C₁-C₈acyl or by C₁-C₁₂alkylaminocarbonyl; or 1,2-phenylene, 2-benzylene, 1,2-, 2,3- or 1,8-naphthylene, the aforementioned groups each being unsubstituted or substituted independently of the others by one or more groups, e.g. by C₁-C₆alkyl; and

R₁₃ is linear C₂alkylene that is unsubstituted or substituted by C₁-C₆alkyl, C₃-C₆cycloalkyl, phenyl, naphthyl or by benzyl; 1,2-cycloalkylene or -cycloalkenylene, -bicycloalkylene or -bicycloalkenylene having from 4 to 10 carbon atoms, each of which is unsubstituted or substituted by one or more groups, e.g. C₁-C₆alkyl, phenyl or by benzyl; 3,4-pyrrolidinylene the nitrogen atom of which is substituted by hydrogen, phenyl, benzyl, C₁-C₁₂alkoxycarbonyl or by C₁-C₁₂alkylaminocarbonyl; or 1,2-phenylene that is unsubstituted or substituted by C₁-C₆alkyl, or is a radical, less two hydroxy groups in the ortho positions, of a mono- or di-saccharide; and

R_{c} is hydrogen, C₁-C₆alkyl, phenyl or benzyl.

R₉ however, preferably is an optionally substituted ferrocenyl radical, e.g. a radical of the formulae

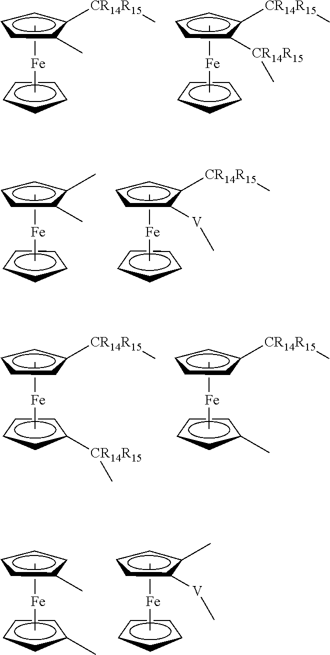

wherein R₁₄ and R₁₅ independently of one another, each represent hydrogen, C₁-C₂₀alkyl, C₁-C₄fluoroalkyl, C₃-C₈cycloalkyl, C₆-C₁₂aryl or C₃-C₁₂heteroaryl having heteroatoms selected from the group consisting of O, S and N, the aforementioned groups being unsubstituted or substituted independently of the others by one or more substituents e.g. by halogen, OR₀₀, NR₀₇R₀₈, C₁-C₆alkyl, C₁-C₆alkoxy, C₃-C₈cycloalkyl, C₃-C₈cycloalkoxy, phenyl, C₁-C₆alkylphenyl, C₁-C₆alkoxy phenyl, C₃-C₈heteroaryl or halogen; or one of

R₁₄ or R₁₅ is connected through a bridging group with the adjacent phosphor atom in the secondary phosphine group of which the carbon atom bearing R₁₄ and R₁₅ is attached to (i.e. the phosphor atom in the group R₇R₈P— or —PR₁₀R₁₁), e.g. via a C₁-C₈alkylene, alkenylene, or alkynylene bridge optionally with one or more of the carbon atoms substituted with a heteroatom, said bridge optionally being substituted with one or more substituents that may be selected among C₁-C₁₂alkyl, C₃-C₈cycloalkyl, C₆-C₁₂aryl or C₃-C₁₂heteroaryl having heteroatoms selected from the group consisting of O, S and N, the aforementioned groups being unsubstituted or substituted independently of the others by one or more substituents e.g. by C₁-C₆alkyl, C₁-C₆alkoxy, C₃-C₈cycloalkyl, C₃-C₈cycloalkoxy, phenyl, C₁-C₆alkylphenyl, C₁-C₆alkoxy phenyl or C₃-C₈heteroaryl; and wherein the cyclopentadienyl rings, independently of one another, may be substituted by one or more substituents, e.g. by a halogen atom, additional secondary phosphine groups as described herein, or a substituent bound to the cyclopentadienyl ring via a C atom, S atom, Si atom, a P(O) group or a P(S) group all of which may carry one or more secondary phosphine groups as herein described; examples of such substituents on the cyclopentadienyl rings are methyl, ethyl, n- and i-propyl, n-, i- and t-butyl, pentyl, hexyl, cyclohexyl, cyclohexyl methyl, phenyl, benzyl, trimethylsilyl, F, Cl, Br, methylthio, methylsulphonyl, methylsulphoxyl, phenylthio, phenylsulphonyl, phenylsulphoxyl, —CH(O), —C(O)OH, —C(O)—OCH₃, —C(O)—OCH₂H₅, —C(O)—NH₂, —C(O)—NHCH₃, —C(O)—N(CH₃)₂, —SO₃H, —S(O)—OCH₃, —S(O)—OC₂H₅, —S(O)₂—OCH₃, —S(O)₂—OC₂H₅, —S(O)—NH₂, —S(O)—NHCH₃, —S(O)—N(CH₃)₂, —S(O)—NH₂, —S(O)₂—NHCH₃, —S(O)₂—N(CH₃)₂, —P(OH)₂, PO(OH)₂, —P(OCH₃)₂, —P(OCH₃)₂, —PO(OCH₃)₂, —PO(OC₂H₅)₂, trifluoromethyl, methylcyclohexyl, methylcyclohexylmethyl, methylphenyl, dimethylphenyl, methoxyphenyl, dimethoxyphenyl, hydroxymethyl, hydroxyethyl, hydroxypropyl, —CH₂NH₂, —CH₂N(CH₃)₂, —CH₂CH₂NH₂, —CH₂CH₂N(CH₃)₂, methoxymethyl, ethoxymethyl, methoxyethyl, ethoxyethyl, HS—CH₂—, HS—CH₂CH₂—, CH₃S—CH₂—, CH₃S—CH₂CH₂—, —CH₂—C(O)OH, —CH₂CH₂—C(O)OH, —CH₂—C(O)OCH₃, —CH₂CH₂—C(O)OCH₃, —CH₂—C(O)NH₂, —CH₂CH₂—, C(O)NH₂, —CH₂—C(O)—N(CH₃)₂, —CH₂—SO₃H, —CH₂CH₂—C(O)N(CH₃)₂, —CH₂CH₂—SO₃H, —CH₂—SO₃CH₃, —CH₂CH₂—SO₃CH₃, —CH₂—SO₂NH₂, —CH₂—SO₂N(CH₃)₂, —CH₂—PO₃H₂, —CH₂CH₂—PO₃H ₂, —CH₂—PO(OCH₃), —CH₂CH₂—PO(OCH₃)₂, —C₆H₄—C(O)OH, —C₆H₄—C(O)OCH₃, —C₆H₄—S(O)₂OH, —C₆H₄—S(O)₂OCH₃, —CH₂—O—C(O)CH₃, —CH₂CH₂—O—C(O)CH₃, —CH₂—NH—C(O)CH₃, —CH₂CH₂—NH—C(O)CH₃, —CH₂—O—S(O)₂CH₃, —CH₂CH₂—O—S(O)₂CH₃, —CH₂—NH—S(O)₂CH₃, —CH₂CH₂—NH—S(O)₂CH₃, —P(O)(C₁-C₈alkyl)₂, —P(S)(C₁-C₈alkyl)₂, —P(O)(C₆-C₁₀aryl)₂, —P(S)(C₆-C₁₀aryl)₂, —C(O)—C₁-C₈alkyl and —C(O)—C₆-C₁₀aryl.

V represents an optionally substituted C₆-C₂₀ arylene or C₃-C₁₆ heteroarylene group wherein the connecting bonds to such group are positioned ortho to one another, i.e. on adjacent carbon atoms in the ring structure. An arylene group V preferably contains from 6 to 14 carbon atoms. Examples of arylene are phenylene, naphthylene, anthracylene and phenanthrylene. Preference is given to phenylene and naphthylene. A heteroarylene group V preferably contains from 5 to 14 carbon atoms. The heteroatoms are preferably selected from the group consisting of O, S and N. The heteroarylene can contain from 1 to 4, preferably 1 or 2, identical or different heteroatoms. A few examples are pyridinylene, pyrimidinylene, pyrazinylene, pyrrolylene, furanylene, oxazolylene, imidazolylene, benzofuranylene, indolylene, benzimidazolylene, quinolylene, isoquinolylene, quinazolinylene and quinoxalinylene. When substituted the arylene or heteroarylene group may be substituted by one or more identical or different groups e.g. C₁-C₄alkyl, C₁-C₄alkoxy, C₁-C₄fluoroalkyl or C₁-C₄fluoroalkoxy.

R₀₀ represents H, C₁-C₁₂alkyl, C₁-C₁₂alkenyl, C₁-C₁₂alkynyl, C₃-C₈cycloalkyl, C₆-C₁₂aryl, R₀₁R₀₂R₀₃Si, C₁-C₁₈acyl that is optionally substituted, e.g. by halogen, hydroxy, C₁-C₈alkoxy or R₀₄R₀₅N—, or R₀₀ represents R₀₆—X₀₁—C(O)—; R₀₁, R₀₂ and R₀₃ are each, independently of one another, C₁-C₁₂alkyl, unsubstituted or C₁-C₄alkyl, C₁-C₄haloalkyl or C₁-C₄alkoxy-substituted C₆-C₁₀aryl or C₇-C₁₂aralkyl; R₀₄ and R₀₅ are each, independently of one another, hydrogen, C₁-C₁₂alkyl, C₃-C₈cycloalkyl, C₆-C₁₀aryl or C₇-C₁₂-aralkyl, or R₀₄ and R₀₅ together are trimethylene, tetramethylene, pentamethylene or 3-oxapentylene; R₀₆ is C₁-C₁₈alkyl, unsubstituted or C₁-C₄alkyl- or C₁-C₄alkoxy-substituted C₃-C₈cycloalkyl, C₆-C₁₀aryl or C₇-C₁₂aralkyl; X₀₁ is —O— or —NH—; all the aforementioned groups being unsubstituted or substituted independently of the others by one or more substituents e.g. by halogen, C₁-C₆alkyl, C₁-C₆alkoxy, C₃-C₈cycloalkyl, C₃-C₈cycloalkoxy, phenyl, C₁-C₆alkyl phenyl, C₁-C₆alkoxy phenyl, C₃-C₈heteroaryl.

R₀₇ and R₀₈ independently of one another represents hydrogen, C₁-C₁₂alkyl, C₃-C₈cycloalkyl, C₆-C₁₀aryl or C₇-C₁₂aralkyl, or R₀₇ and R₀₈ together are trimethylene, tetramethylene, pentamethylene or 3-oxapentylene; the aforementioned groups being unsubstituted or substituted independently of the others by one or more substituents e.g. by halogen, C₁-C₆alkyl, C₁-C₆alkoxy, C₃-C₈cycloalkyl, C₃-C₈cycloalkoxy, phenyl, C₁-C₆alkyl phenyl, C₁-C₆alkoxy phenyl, C₃-C₈heteroaryl.

Alkyl groups, e.g. R₀₁, R₀₂ and R₀₃, can be linear or branched and the alkyl preferably has from 1 to 12 carbon atoms, particularly preferably from 1 to 4 carbon atoms. Aryl groups R₀₁, R₀₂ and R₀₃ can be, for example, phenyl or naphthyl and aralkyl groups R₀₁, R₀₂, and R₀₃ can be benzyl or phenylethyl. Some examples of R₀₁, R₀₂ and R₀₃ are methyl, ethyl, n- or i-propyl, n-, i- or t-butyl, pentyl, hexyl, heptyl, octyl, nonyl, decyl, undecyl, dodecyl, phenyl, benzyl, methylphenyl, methylbenzyl, methoxyphenyl, dimethoxyphenyl and methoxybenzyl.

Some preferred examples of silyl groups R₀₁R₀₂R₀₃Si are trimethylsilyl, tri-n-butylsilyl, t-butyldimethylsilyl, 2,2,4,4,-tetramethylbut-4-yl-yldimethylsilyl, triphenylsilyl and tri-i-propylsilyl

In a preferred embodiment, R₀₄ and R₀₅ are each, independently of one another, hydrogen, C₁-C₄alkyl, C₅-C₆cycloalkyl, phenyl or benzyl, or R₀₄ and R₀₅ together are tetramethylene, pentamethylene or 3-oxapentyl-1,5-ene. The substituent C₁-C₈alkoxy is preferably C₁-C₄alkoxy such as methoxy, ethoxy, propoxy or butoxy.

An acyl group, e.g. as defined for R₀₀, preferably has from 1 to 12 carbon atoms, particularly preferably from 1 to 8 carbon atoms, and is, in particular, derived from a carboxylic acid. Examples of such carboxylic acids are aliphatic, cycloaliphatic and aromatic carboxylic acids having from 1 to 18 carbon atoms, preferably from 1 to 12 carbon atoms. Some examples of acyl are acetyl, propanoyl, butanoyl, pentanoyl, hexanoyl, octanoyl, dodecanoyl, tetradecanoyl, octadecanoyl, cyclohexylcarbonyl, benzoyl, methylbenzoyl, phenylacetyl, pyridylcarbonyl, naphthylcarbonyl. Some examples of substituted acyl are groups of the formula R₀₉—C(O)—, where R₀₉ is hydroxymethyl, methoxymethyl, ethoxymethyl, 2-hydroxyeth-1-yl, 2-methoxyeth-1-yl, hydroxypropanoyl, fluoromethyl, chloromethyl, difluoromethyl, dichloromethyl, trifluoromethyl, trichloromethyl, aminomethyl, methylaminomethyl, dimethylaminomethyl, 1-aminoeth-1-yl, 1-methylaminoeth-1-yl, 1-dimethylaminoeth-1-yl, 2-aminoeth-1-yl, 3-aminoprop-1-yl, 4-aminobut-1-yl, pyrrolinyl-N-methyl, piperidinyl-N-methyl, morpholino-N-methyl, 4-amino-cyclohex-1-yl, methoxyphenyl, hydroxyphenyl, aminophenyl, dimethylaminophenyl, hydroxybenzyl, p-aminobenzyl and p-dimethylaminobenzyl.

An alkyl group R₀₆ has from 1 to 12 carbon atoms, particularly preferably from 1 to 8 carbon atoms. The alkyl can be linear or branched. A cycloalkyl group R₀₆ is preferably cydopentyl or cyclohexyl. An aryl group R₀₆ can be naphthyl or in particular phenyl. An aralkyl group R₀₆ can be phenylethyl or in particular benzyl. Some examples of R₀₆ are methyl, ethyl, n- or i-propyl, n-, i- or t-butyl, pentyl, hexyl, heptyl, octyl, nonyl, decyl, undecyl, dodecyl, cydopentyl, cyclohexyl, methylcyclohexyl, phenyl, benzyl, methylphenyl, methylbenzyl, methoxyphenyl, dimethoxyphenyl and methoxybenzyl.

In a preferred embodiment, R₀₇ and R₀₈ are each, independently of one another, hydrogen, C₁-C₄alkyl, C₅-C₆cycloalkyl, phenyl or benzyl, or R₀₇ and R₀₈ together are tetramethylene, pentamethylene or 3-oxapentyl-1,5-ene.

A preferred subgroup of diphosphines Y is formed by those of the formulae

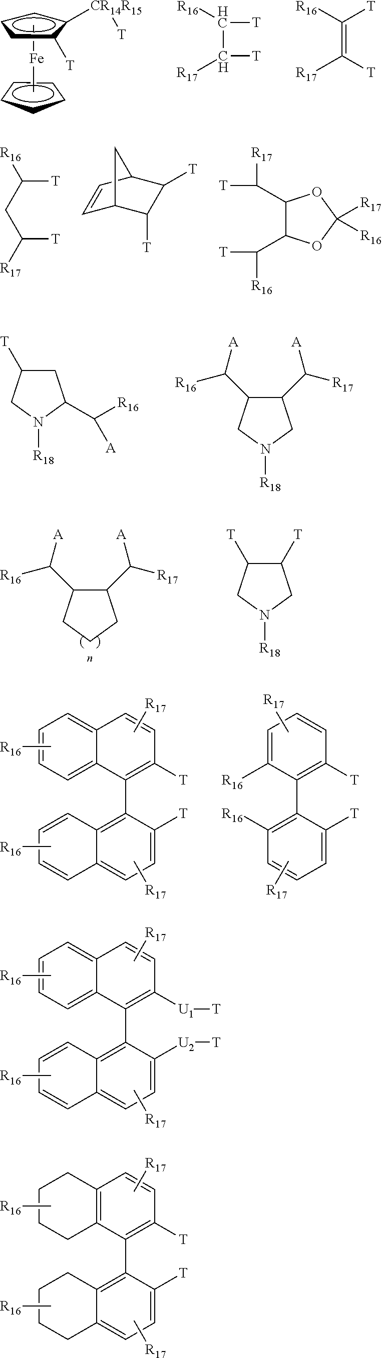

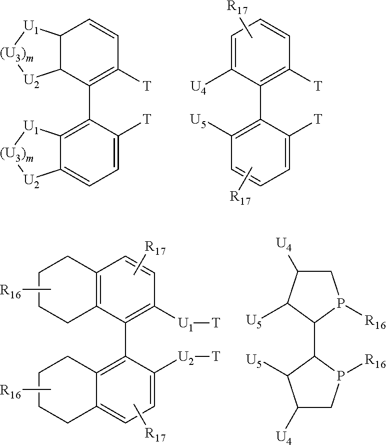

wherein

U₁, U₂ independently are CH₂, O, NR₁₈;

U₃ denotes CH₂, CF₂;

U₄ and U₅ are independently of the other R₁₆ or together represents a ring having from 3 to 8 ring carbon atoms, being optionally heterocyclic having from 3 to 8 ring atoms and 1 or 2 hetero atoms from the group O, S and N/NR₁₈;

m independently for each U₃ is 1 or 2;

n is 0, 1 or 2;

R₁₆ and R₁₇ are each independently of the other hydrogen, C₁-C₆alkyl, C₁-C₆alkoxy, halogen, phenyl, benzyl, Si(R₁₄)₃, COOR₁₄, CN, C₁-C₆alkyn, or phenyl or benzyl having from 1 to 3 C₁-C₄alkyl or C₁-C₄alkoxy substituents, or R₁₆ and R₁₇ together represents an C₃-C₈alkylene, alkenylene, or alkynylene bridge optionally with one or more of the carbon atoms substituted with a heteroatom preferably selected from the group consisting of O, S and NR₆/N, said bridge optionally being substituted with one or more substituents, e.g. selected among C₁-C₁₇alkyl, C₃-C₈cycloalkyl, C₆-C₁₂aryl or C₃-C₁₂heteroaryl having heteroatoms selected from the group consisting of O, S and N, the aforementioned groups being unsubstituted or substituted independently of the others by one or more substituents e.g. by C₁-C₆alkyl, C₁-C₆alkoxy, C₃-C₈cycloalkyl, C₃-C₈cycloalkoxy, phenyl, C₁-C₆alkylphenyl, C₁-C₆alkoxy phenyl or C₃-C₈heteroaryl.

R₁₄ and R₁₅ are as previously defined;

R₁₈ is hydrogen, C₁-C₆alkyl, phenyl, benzyl, C₁-C₆alkoxy-CO—, phenyl-CO—, C₁-C₆alkyl-CO—, naphthyl-CO— or C₁-C₆alkyl-NH—CO—;

T may be identical or different groups —P(R₇R₈), wherein R₇ and R₈ are as defined previously and preferably are different or identical and each preferably represents C₁-C₆alkyl, C ₁-C₆alkoxy, C₅-C₆cycloalkyl, phenyl, benzyl or phenyl or benzyl having from l to 3 C₁-C₆alkyl, C₁-C₆alkoxy, —CF₃ or partially or fully fluorinated C₁-C₆alkoxy substituents; or R₇ and R₈ may form a 4-8 member ring such as an alkylene bridge having from 4 to 8 carbon atoms optionally with one or more of the carbon atoms substituted with a heteroatom preferably selected from the group consisting of O, S and NR₆/N, said bridge optionally being substituted with one or more substituents, e.g. selected among C₁-C₁2alkyl, C₃-C₈cycloalkyl, C₆-C₁₂aryl or C₃-C₁₂heteroaryl having heteroatoms preferably selected from the group consisting of O, S and N, the aforementioned groups being unsubstituted or substituted independently of the others by one or more substituents e.g. by C₁-C₆alkyl, C₁-C₆alkoxy, C₃-C₈cycloalkyl, C₃-C₈cycloalkoxy, phenyl, C₁-C₆alkylphenyl, C₁-C₆alkoxy phenyl or C₃-C₈heteroaryl

Of those diphosphines, chirally substituted compounds are especially preferred.

Some preferred examples of diphosphines Y are as follows (Ph is phenyl):

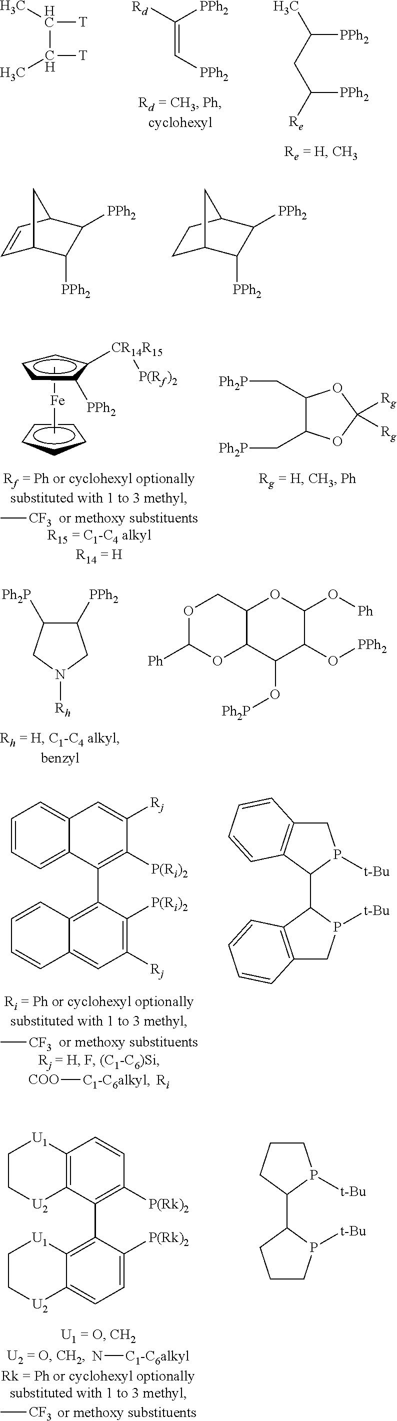

Suitable diphosphines and diphosphinites have been described, for example, by H. B. Kagan in Chiral Ligands for Asymmetrie Catalysis, Asymmetrie Synthesis, Volume 5, pp. 13-23, Academic Press, Inc., N. Y. (1985). The preparation of ferrocenyl diphosphine ligands is described, for example, in U.S. Pat. No. 5,463,097; U.S. Pat. No. 5,565,594; U.S. Pat. No. 5,583,241; U.S. Pat. No. 5,563,309; U.S. Pat. No. 5,627,293; U.S. Pat. No. 6,015,919; U.S. Pat. No. 6,169,192; U.S. Pat. No. 6,191,284, U.S. Pat. No. 6,515,183; U.S. Pat. No. 6,777,567; U.S. Pat. No. 6,828,271; U.S. Pat. No. 7,375,241; and International patent applications nos. WO 2001/04131-A 1; WO 2005/108409-A2; WO 2006/003194-A 1; WO 2006/003195-A 1; WO 2006/003196-A 1 ; WO 2006/114438-A2; WO 2006/117369-A 1; WO 2007/017522-A2 and WO 2007/020221-A2.

A⁻ can be derived from inorganic or organic oxy acids. Examples include those wherein A is the anion of an organic oxy acid that contains a group C(═O)O, S(═O)O or P(═O)O in the anion. The organic oxy acid may be mono- or poly-basic, for example mono- or di-basic. Monobasic acids are especially preferred; in the case of polybasic acids, the excess acidic OH groups may be blocked, for example by esterification. The organic oxy acid may be, for example, a partial ester of an at least dibasic inorganic oxy acid, preferably those of formula R₁₀₀—OSO₂—OH or (R₁₀₀—O)₂P(O)—OH wherein R₁₀₀ is the monovalent radical of an aliphatic, cycloaliphatic, cycloaliphatic-aliphatic, aromatic or araliphatic alcohol having from 1 to 20 carbon atoms, preferably from 1 to 12 carbon atoms and especially from 1 to 8 carbon atoms. R₁₀₀ may be, for example, branched and, preferably, linear C₁₋₂₀alkyl, preferably C₁₋₁₂alkyl and especially C₁₋₈alkyl. Some examples are methyl, ethyl, propyl, butyl, pentyl, hexyl, octyl, decyl and dodecyl. R₁₀₀ may be, for example, C₃₋₈cycloalkyl and preferably C₅₋₆cycloalkyl. Some examples are cyclopropyl, cyclobutyl, cyclopentyl, cyclohexyl, cycloheptyl and cyclooctyl. R₁₀₀ may be, for example, C₃₋₈cycloalkyl-(CH₂)ₚ— and preferably C₅₋₆cycloalkyl-(CH₂)ₚ—, wherein p is a number from 1 to 4 and is preferably 1 or 2. Some examples are cyclopropyl-CH₂—, cyclobutyl-CH₂—, cyclopentyl-CH₂—, cyclohexyl-CH₂—, cycloheptyl-CH₂—, cyclooctyl-CH₂—, cyclopropyl-CH₂—CH₂—, cyclobutyl-CH₂-CH₂—, cyclopentyl-CH₂—CH₂—, cyclohexyl-CH₂—CH₂—, cycloheptyl-CH₂—CH₂—, cyclooctyl-CH₂'1CH₂—. R₁₀₀ may be, for example, C₆₋₁₆aryl, preferably C₆₋₁₀aryl and especially phenyl. R₁₀₀ may be, for example, optionally substituted C₆-C₂₀arylene e.g. C₆₋₁₆aryl-(CH₂)ₚ—, preferably C₆₋₁₀aryl-(CH₂)ₚ— and especially phenyl-(CH₂)ₚ—, wherein p is a number from 1 to 4 and is preferably 1 or 2. Some examples are benzyl, phenylethyl and naphthylmethyl.

The organic oxy acid from which A⁻ is derived is preferably of formula R₁₀₁—S(O)ₖ—OH; R₁₀₁—(R₁₀₀O)ₗP(O)—OH or R₁₀₂—C(O)—OH wherein k is 1 or 2, l is 0 or 1, R₁₀₀ is the monovalent radical of an aliphatic, cycloaliphatic, cycloaliphatic-aliphatic, aromatic or araliphatic alcohol having from 1 to 20 carbon atoms, preferably from 1 to 12 carbon atoms and especially from 1 to 8 carbon atoms; R₁₀₁ is an aliphatic, cycloaliphatic, cycloaliphatic-aliphatic, aromatic or araliphatic radical having from 1 to 20 carbon atoms, preferably from 1 to 12 carbon atoms and especially from 1 to 8 carbon atoms, that is unsubstituted or mono- or poly-substituted by C₁₋₆alkyl, C₁₋₆hydroxyalkyl, C₁₋₆haloalkyl (especially fluoro- or chloro-alkyl), C₁₋₆alkoxy, C₁₋₆alkylthio, C₅₋₆cycloalkyl, C₆₋₁₀aryl, —OH, —F, —Cl, —Br, —CN, —NO₂ or by —C(O)O—C₁₋₆alkyl, it being possible for the substituents cycloalkyl and aryl to be substituted by C₁₋₆alkyl, C₁₋₆alkoxy, —OH, —F, —Cl, —Br, —CN, —NO₂, C₁₋₆haloalkyl or by —C(O)O—C₁₋₆alkyl; and R₁₀₂ is hydrogen or has independently the same meaning as given for R₁₀₁. For R₁₀₀, the examples and preferences mentioned hereinbefore apply. R₁₀₁ and R₁₀₂ may be, for example, branched and, preferably, linear C₁₋₂₀alkyl, preferably C₁₋₁₂alkyl and especially C₁₋₈alkyl. Some examples are methyl, ethyl, propyl, butyl, pentyl, hexyl, octyl, decyl and dodecyl. R₁₀₁ and R₁₀₂ may be, for example, C₃₋₈cycloalkyl and preferably C₅₋₆-cycloalkyl. Some examples are cyclopropyl, cyclobutyl, cyclopentyl, cyclohexyl, cycloheptyl and cyclooctyl. R₁₀₁ and R₁₀₂ may be, for example, C₃₋₈cycloalkyl-(CH₂)ₚ— and preferably C5-6-cycloalkyl-(CH₂)ₚ—, wherein p is a number from 1 to 4 and is preferably 1 or 2. Some examples are cyclopropyl-CH₂—, cyclobutyl-CH₂—, cyclopentyl-CH₂—, cyclohexyl-CH₂—, cycloheptyl-CH₂—, cyclooctyl-CH₂—, cyclopropyl-CH₂—CH₂—, cyclobutyl-CH₂—CH₂—, cyclopentyl-CH₂—CH₂—, cyclohexyl-CH₂—CH₂—, cycloheptyl-CH₂—CH₂— and cyclooctyl-CH₂-CH₂—. R₁₀₁ and R₁₀₂ may be, for example, C₆₋₁₆aryl, preferably C₆₋₁₀ aryl and especially phenyl. R₁₀₁ and R₁₀₂ may be, for example, optionally substituted C₆-C₂₀arylene e.g. C₆₋₁₆aryl-(CH₂)ₚ—, preferably C₆₋₁₀aryl-(CH₂)ₚ— and especially phenyl-(CH₂)ₚ—, wherein p is a number from 1 to 4 and is preferably 1 or 2. Some examples are benzyl, phenylethyl and naphthylmethyl.

In a preferred form, the organic acids from which A⁻ is derived are of formulae R₁₀₁—S(O)₂—OH and R₁₀₁—C(O)—OH wherein R₁₀₁ is unsubstituted or C₁₋₄alkyl, C₁₋₄alkoxy, —OH, —F, —Cl—, —Br, —NO₂, —C(O)O—C₁₋₄alkyl, cyclopentyl-, cyclohexyl- or phenyl-substituted C₁₋₆alkyl, preferably C₁₋₄alkyl, C₅₋₆-cycloalkyl or phenyl, it being possible for the substituents cyclopentyl, cyclohexyl or phenyl to be substituted by C₁₋₄alkyl, C₁₋₄alkoxy, —F, —Cl, —Br or by C₁₋₄haloalkyl.

Some examples of preferred organic acids are acetic acid, propionic acid, butyric acid, mono-, di- or tri-chloro- or mono-, di- or tri-fluoro-acetic acid, perfluoropropionic acid, cyclohexanecarboxylic acid, benzoic acid, mono-, di- or tri-methylbenzoic acid, fluoro- or chloro-benzoic acid, trifluoromethylbenzoic acid, phenylacetic acid, methanesulfonic acid, ethanesulfonic acid, propanesulfonic acid, butanesulfonic acid, mono-, di- or tri-chloro- or mono-, di- or tri-fluoro-methanesulfonic acid, benzenesulfonic acid, mono-, di- or tri-methylbenzenesulfonic acid and fluoro- or chloro-benzenesulfonic acid.

Complex acids from which A⁻ can be derived are, for example, the halo complex acids of the elements B, P, As, Sb and Bi.

A⁻ in formula (IVa) can be derived from inorganic or organic oxy acids. Examples of such acids are H₂SO₄, HClO₄, HClO₃, HBrO₄, HIO₄, HNO₃, H₃PO₃, H₃PO₄, CF₃SO₃H, C₆H₅SO₃H, CF₃COOH and CCl₃COOH. Preferred examples of A⁻ derived from complex acids in formula (IVa) are ClO₄⁻, CF₃SO₃⁻, BF₄⁻, B(phenyl)₄⁻, PF₆⁻, SbCl₆⁻, AsF₆⁻ and SbF₆⁻.

M⁺ in formula (IVb) when a phosphonium cation, it may be, for example R_{w}RₓR_{y}R_{z}P⁺, wherein R_{w}, Rₓ, R_{y}, R_{z} independently of one another can be hydrogen, halogen, linear or branched C₁-C₆alkyl, C₃-C₁₂-cycloalkyl, substituted or unsubstituted C₆-C₁₂aryl, substituted or unsubstituted C₄-C₁₂heteroaryl. Two of R₂, Rₓ, R_{y}, R_{z} can build a ring. R_{w}, Rₓ, R_{y}, R_{z} can also contain a polycyclic structure, like for example adamantyl substituents. R_{w}, Rₓ, R_{y}, R_{z} independently from each one can contain at least one chiral centre or they can be different and the chirality resides in the phosphorous atom, which can then be used as single enantimoer or as a mixture of enantiomers. When M⁺ is an alkali metal cation, it may be, for example, a Li, Na, K, Rb or a Cs cation. When M⁺ is quaternary ammonium, it may be, for example R′_{w}R′ₓR′_{y}R′_{z}N⁺, and it may contain a total of from 1 to 40, preferably from 4 to 24, carbon atoms. R′₂, R′ₓ, R′_{y}, R′_{z} independently of one another can be hydrogen, linear or branched C₁-C₆alkyl, C₃-C₁₂cycloalkyl, substituted or unsubstituted C₆-C₁₂aryl, substituted or unsubstituted C₄-C₁₂heteroaryl. Two of R′_{w}, R′ₓ, R′_{y}, R′_{z} can build a ring. R′_{w}, R′ₓ, R′_{y}, R′_{z} can also contain a polycyclic structure. M⁺ may correspond to the formulae phenyl-N⁺(C₁-C₆alkyl)₃, benzyl-N⁺(C₁-C₆alkyl)₃ or (C₁-C₆alkyl)₄N³⁰. Preferably m⁺ is Li⁺, Na⁺ or K⁺or (C₁-C₆alkyl)₄N+.

Z in formula (IV) is preferably Br or Cl and especially Cl. Z in formula (IVb), (IVe), (IVf) and (IVg) is preferably Br or I and Z in formulae (IVc) and (IVd) is preferably 1.

Especially suitable diphosphine ligands which can preferably be used in iridium based catalysts of formula (IV) are, for example:

{(R)-1-[(S)-2-diphenylphosphino)ferrocenyl]}ethyl-di(3,5-dimethyl-4-N,N-dipropyl-aminophenyl)phosphine

{(R)-1-[(S)-2-diphenylphosphino)ferrocenyl]}ethyl-di(3,5-diisopropyl-4-N,N-dimethyl-aminophenyl)phosphine

{(R)-1-[(S)-2-diphenylphosphino)ferrocenyl]}ethyl-di(3,5-diisopropyl-4-N,N-dibenzylyl-aminophenyl)phosphine

{(R)-1-[(S)-2-diphenylphosphino)ferrocenyl]}ethyl-di(3,5-dimethyl-4-N,N-dibenzylyl-aminophenyl)phosphine

{(R)-1-[(S)-2-diphenylphosphino)ferrocenyl]}ethyl-di(3,5-dimethyl-4-(1′-pyrrolo)-phenyl)phosphine

{(R)-1-[(S)-2-diphenylphosphino)ferrocenyl]}ethyl-di(3,5-dimethyl-4-N,N-dipentyl-amino-phenyl)phosphine

{(R)-1-[(S)-2-diphenylphosphino)ferrocenyl]}ethyl-di(3,5-dimethyl-4-N,N-dimethyl-aminophenyl)phosphine 1,4-bis(diphenylphosphino)butane

{(R)-1-[(S)-2-di(4-methoxyphenyl)phosphino)ferrocenyl]}ethyl-di(3,5-dimethyl-4-N,N-dimethylaminophenyl)phosphine

{(R)-1-[(S)-2-diphenylphosphino)ferrocenyl]}ethyl-di(3,5-dimethyl-phenyl)phosphine—also referred to as xyliphos

Compounds comprising a carbon-halogen bond suitable used as co-catalysts in a process according to the present invention may be selected among those compounds of formula (VI)

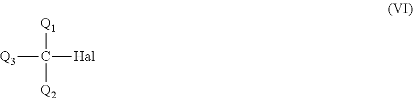

wherein Hal represents a halogen atom; and Q₁, Q₂, and Q₃ are each independently of the other a group selected among H, linear or branched C₁-C₁₂alkyl, C₂-C₁₂alkenyl or C₂-C₁₂alkynyl, C₃-C₈cycloalkyl, heterocycloalkyl bonded via a carbon atom and having from 3 to 8 ring atoms and 1, 2 or 3 hetero atoms preferably selected from the group consisting of O, S and N12.₁₉; or is a group selected among C₇-C₁₆aralkyl bonded via an alkyl carbon atom, C₁-C₁₂, alkyl substituted by the mentioned C₃-C₈cycloalkyl, heterocycloalkyl or C₃-C₁₁heteroaryl bonded via a ring carbon atom and having 1, 2 or 3 hetero atoms in the ring selected from the group consisting of O, S and N/NR₁₉; or is a group selected among C₆-C₁₂aryl, or C₃-C₁₁heteroaryl bonded via a ring carbon atom and having 1, 2 or 3 hetero atoms in the ring preferably selected from the group consisting of O, S and N; the aforementioned groups being unsubstituted or substituted independently of the others by one or more substituents e.g. —CN, —NO₂, F, Cl, Br, I, C₁C₁₂alkyl, C₁-C₁₂alkoxy, C₁-C₁₂alkylthio, C₁-C₁₂haloalkyl, —OH, C₆-C₁₂aryl or -aryloxy or -arylthio, C₇-C₁₆oralkyl or -aralkoxy or -aralkylthio, secondary amino having from 2 to 24. carbon atoms, —CONR₁₉R₂₀ or by —COOR₁₉; or

Q₂ and Q₃ together represents a group ═O, ═S, ═NR₁₉, ═CQ₂Q₃ where Q₂ and R₁₉ are as previously defined, or

Q₁, Q₂ and Q₃ together represents a group ≡CQ₁ where Q1 is as previously defined; or

Q₁, Q₂, and Q₃ together form, with the carbon atom to which they are attached, a ring having from 3 to 16 ring carbon atoms, being optionally heterocyclic having from 3 to 16 ring atoms and 1, 2 or 3 (or more) hetero atoms preferably selected from the group consisting of O, S and N/NR₁₉, said ring optionally being substituted e.g. with —CN, —NO₂, halogen, C₁-C₁₂alkyl, C₁-C₁₂alkoxy, C₁-C₁₂alkylthio, C₁-C₁₂haloalkyl, —OH, C₆-C₁₂aryl or -aryloxy or -arylthio, C₇-C₁₆aralkyl or -aralkoxy or -aralkylthio, secondary amino having from 2 to 24 carbon atoms, —CONR₁₉R₂₀ or by —COOR₁₉; or

R₁₉ and R₂₀ are each independently of the other hydrogen, C₁-C₁₂alkyl, phenyl or benzyl.

Compounds of the formula (VI) may comprise additional halogen substituents identical or different from Hal.

When Q₁, Q₂, and Q₃ together form, with the carbon atom to which they are attached, a ring such ring may comprise one or more double bonds and may also be aromatic or heteroaromatic and in either case include bi- or tri-cyclic structures. Preferably Q₁, Q₂, and Q₃ together with the carbon atom to which they are attached form a phenyl ring, being optionally heterocyclic having 1, 2 or 3 hetero atoms, optionally being substituted by one or more substituents, e.g. —CN, —NO², halogen, C₁-C₁₂alkyl, C₁-C₁₂alkoxy, C₁-C₁₂alkylthio, C₁-C₁₂haloalkyl, —OH, C₆-C₁₂-aryl or -aryloxy or -arylthio, C₇-C₁₆-aralkyl or -aralkoxy or -aralkythio, secondary amino having from 2 to 24 carbon atoms, —CONR₁₉R₂₀ or by —COOR₁₉.

Examples of substituents as mentioned above are as already described herein for R₁, R₂ and R₃.

Preferably Hal represents I, Cl or Br, most preferably I or Br; Q₁ represents linear or branched C₁-C₁₂alkyl or C₂-C₁₂alkenyl, C₆-C₁₂aryl or C₃-C₁₁heteroaryl bonded via a ring carbon atom and having 1, 2 or 3 hetero atoms in the ring, or C₇-C₁₆aralkyl bonded via an alkyl carbon atom; Q₂ represents hydrogen or linear or branched C₁-C₁₂alkyl; Q₃ represents hydrogen or linear or branched C₁-C₁₂alkyl; the aforementioned groups being unsubstituted or substituted independently of the others by one or more substituents; or Q₂ and Q₃ together represents a group O or ═CQ₂Q₃; or Q₁, Q₂, and Q₃ together form, with the carbon atom to which they are bonded, a benzene ring, being optionally heterocyclic having 1, 2 or 3 hetero atoms, and optionally being substituted with one or more substituents such as halogen or C₁-C₆alkyl.

Some preferred examples of compounds of the formula (VI) include, halo-benzene (e.g. iodo benzene or bromo benzene), halo-benzyl (e.g halo benzyl caring up to 5 substituents and including benzyl iodide, benzyl bromide, 2-bromo-benzylbromide, 2,3,4,5,6-Pentafluorobenzylbromide), halo-alkyl (e.g linear or branched halo-alkyl compounds such as halo-butyl compounds optionally carrying one or more substituents e.g. 1-C1-butane or t-butylchloride, i-butylchloride or 1,2-dibromo-butane), halo allyl compounds such as allyl-bromide.

The employed co-catalyst compound(s) comprising a carbon-halogen bond, preferably being selected among chlorides, bromides and iodides, are used preferably in amounts of from 0.0001-10 mol %, preferably from 0.001-5 mol %, more preferably from 0.001-1 mol % and even more preferably from 0.01-1 mol %, based on the imine to be hydrogenated.

The hydrogenation process is carried out as to employ the co-catalyst compound(s) in a catalytic effective amount. By catalytic effective amount is understood an amount whereby the hydrogenation reaction rate and/or turnover number of the iridium based catalyst is increased. Preferably this enhancement of reaction rate and/or turnover is 10% or more, more preferably 20% or more, even more preferably 30% or more or most preferably 40% or more compared to a similar reaction under the same conditions but without a co-catalyst being present.

Thus, an aspect of the present invention relates to a process for hydrogenating a prochiral ketimine in the presence of an effective amount of at least one chiral iridium catalyst and at least one co-catalysts comprising a carbon-halogen bond, the co-catalyst being present in an amount such that a hydrogenation reaction rate and/or turnover number of the chiral iridium catalyst is increased by 10% or more (e.g. as stated above) compared to a similar reaction under same conditions but without the co-catalyst being present, to produce an optical isomer of an amine having an enantiomeric excess higher than 50%, preferably higher than 70% and even more preferably higher than 80%.

Compounds of the formula (VI) are readily available or may be produced according to well known methods.

The process according to the invention may comprise the use of an additional co-catalyst selected among phosphonium halide, metal halide or ammonium halides, the halides preferably selected among chlorides, bromides and iodides. Phosphonium is preferably trialkyl phosphonium halides having from 1 to 40 carbon atoms in the alkyl groups. Special preference is given to diadamantylbutylphosphonium iodide or diadamantylbenzylphosphonium bromide or triphenylisopropylphosphonium iodide or triphenylmethylphosphonium bromide. Other preferred phosphonium salts are triphenylmethylphosphonium bromide, diphenyl isopropyl phosphonium iodide, and triphenyl isopropyl phosphonium iodide. Metal halides include LiCl, LiBr, LiI, NaI, or NaBr. Examples of ammonium halides are tetraalkylammonium halides having 1 to 6 carbon atoms in the alkyl groups and include tetrabutylammonium iodide.

In a preferred embodiment of the present invention, the process is carried out without the addition of any phosphonium-, metal- and/or ammonium-halides.

The reaction can be carried out in the absence or in the presence of inert solvents. Suitable solvents, which can be used alone or as a mixture of solvents, are especially aprotic solvents.

The process according to the invention can be performed without adding an acid. However, it further embraces optionally the additional use of an acid. It may be an inorganic or, preferably, an organic acid. When present, the acid is preferably used in at least the same molar amount as the iridium catalyst (equivalent to catalytic amounts) and can also be used in excess. The excess may even consist in the use of the acid as solvent. Preferably the acid is used from 0.001 to 50%, in particular from 0.1 to 50% by weight, based on the substrate to be hydrogenated. In many cases it can be advantageous to use anhydrous acids.

Some examples of inorganic acids are H₂SO₄, highly concentrated sulfuric acid (oleum), H₃PO₄, orthophosphoric acid, HF, HCl, HBr, HI, HClO₄, HBF₄, HPF₆, HAsF₆, HSbCl₆, HSbF₆ and HB(phenyl)₄. H₂SO₄ is particularly preferred.

Examples of organic acids are aliphatic or aromatic, optionally halogenated (fluorinated or chlorinated) carboxylic acids, sulfonic acids, phosphorus(V) acids (for example phosphonic acids, phosphonous acids) having preferably from 1 to 20, especially from 1 to 12 and more especially from 1 to 6, carbon atoms. The organic acid can also contain at least one chiral center, like tartaric acid or camphorsulfonic acid. Other examples of organic acids are formic acid, acetic acid, propionic acid, butyric acid, benzoic acid, phenylacetic acid, cyclo-hexanecarboxylic acid, chloro- or fluoro-acetic acid, dichloro- or difluoro-acetic acid, trichloro- or trifluoro-acetic acid, chlorobenzoic acid, methanesulfonic acid, ethanesulfonic acid, benzenesulfonic acid, p-toluenesulfonic acid, chlorobenzenesulfonic acid, trifluoromethanesulfonic acid, methyl-phosphonic acid and phenylphosphonic acid. Preferred acids are acetic acid, propionic acid, trifluoroacetic acid, methanesulfonic acid and chloroacetic acid.

It is also possible for acidic ion exchangers of an inorganic or organic nature to be used as the acids, or metal oxides in gel form, like for example SiO₂, GeO₂, B₂O₃, Al₂O₃, TiO₂, ZrO₂ and combinations thereof.

In a preferred embodiment of the present invention, the process is carried out without the addition of an acid.

The preparation of the catalysts is known per se and is described for example in such previous references found herein. The preparation of the catalysts of formula (IV) can be carried out, for example, by reacting a di-iridium complex of the formula [XIrZ], (with X, and Z being as defined herein previously) with a diphosphine, e.g. a diphosphine Y as defined herein. The iridium catalysts can be added to the reaction mixture as isolated compounds. It has proved advantageous, however, to produce the catalyst in situ with or without an inert solvent prior to the reaction and to add the co-catalyst compound comprising a carbon-halogen bond and eventually a portion or all of the optional acid present in the hydrogenation reaction media.

The iridium based catalysts are preferably used in amounts of from 0.0001 to 10 mol %, especially from 0.0005 to 10 mol %, and more especially from 0.001 to 10 mol %, based on the imine.

The process is carried out preferably at a temperature of from −20 to 100° C., especially from 0 to 80° C. and more especially from 10 to 70° C., and preferably at a hydrogen pressure of 2×10⁵ to 1.5×10⁷ Pa (2 to 150 bar), especially 10⁶ to 10⁷ Pa (10 to 100

In detail, the process according to the invention can be carried out by first preparing the catalyst by dissolving, for example, (Ir-dieneCl), in a inert solvent, adding a diphosphine and stirring the mixture and optionally adding further reagents such as additional co-catalysts that may have a positive effect on the desired catalytic properties and selectivity. (Ir-dieneCl)₂, can also be used in solid form. A compound comprising a carbon-halogen bond and optionally an acid is added to a solution of imine in an autoclave and the above catalyst solution is added (or vice versa). Hydrogen pressure is applied, thus removing the protective gas that is advantageously used. It is advantageous to ensure that the catalyst solution stands for only a short time, and to carry out the hydrogenation of the imines as soon as possible after the preparation of the catalyst. The reaction mixture is heated, if desired, and then hydrogenated. Where appropriate, when the reaction has ceased the reaction mixture is cooled and the autoclave is depressurized. The reaction mixture can be removed from the autoclave under pressure with nitrogen and the hydrogenated organic compound can be isolated and purified in a manner known per se, for example by extraction or distillation.

In the case of the hydrogenation of aldimines and ketimines, the aldimines and ketimines can also be formed in situ before or during the hydrogenation. In a preferred form, an amine and an aldehyde or a ketone are mixed together and added to the catalyst solution and the aldimine or ketimine formed in situ is hydrogenated. It is also possible, however, to use an amine, a ketone or an aldehyde together with the catalyst as the initial batch and to add the ketone or the aldehyde or the amine thereto, either all at once or in metered amounts.

The hydrogenation can be carried out continuously or batchwise in various types of reactors. Preference is given to those reactors which allow comparatively good intermixing and good removal of heat, such as for example, loop reactors. That type of reactor has proved to be especially satisfactory when small amounts of catalyst are used.

The process according to the invention yields the corresponding amines in short reaction times while having chemically a high degree of conversion, with surprisingly good optical yields (ee) of 50% or more being obtained even at relatively high temperatures of more than 50° C.

The hydrogenated organic compounds that can be prepared in accordance with the invention, for example the amines, are biologically active substances or are intermediates for the preparation of such substances, especially in the field of the preparation of pharmaceuticals and agrochemicals. For example, O,O-dialkylarylketamine derivatives, especially those having alkyl and/or alkoxyalkyl groups, are effective as pesticides, especially as herbicides. The derivatives may be amine salts, acid amides, for example of chloroacetic acid, tertiary amines and ammonium salts.

Especially important in this connection are the optically active amines of formula (IIa), (IIb) and (IIc)

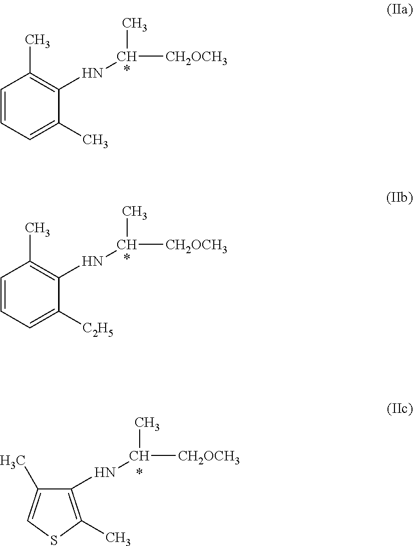

which can be prepared from the imines of formula (Ia), (Ib) and (Ic) respectively using the hydrogenation processes as disclosed herein, and which can be converted in accordance with methods that are customary per se with chloroacetic acid into the herbicides of the chloroacetanilide type.

The above symbol * indicates predominantly one configurational isomer, which means that the enantiomeric excess (ee) is at least 50%, preferably at least 70% and particularly preferably at least 80%.

Thus, the present invention further relates to a process for the preparation of a compound of either formula (IIIa), (IIIb) or (IIIc)

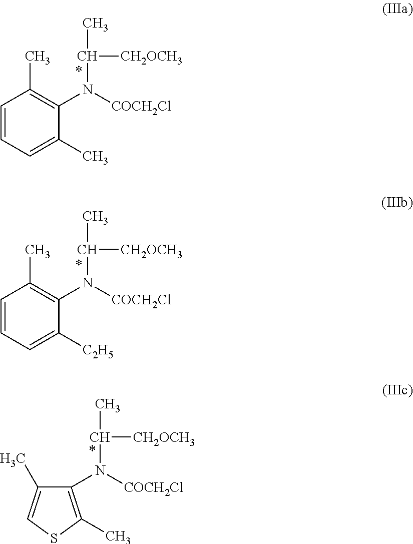

comprising the steps of
i. forming a reaction mixture comprising a) an imine compound of either formula (Ia), (Ib) or (Ic) respectively and optionally an inert solvent, and b) one or more iridium complexes as catalysts, preferably iridium complexes comprising compounds of the formula (VII) as ligands, and one or more co-catalysts selected among compounds comprising a carbon-halogen bond, preferably a co-catalyst selected among compounds of the formula (VI);
ii. reacting the reaction mixture with hydrogen under elevated preassure to form an amine compound of either formulae (11a), (11b) or (IIc) respectively;
iii. reacting the thus formed amine with chloroacetic acid chloride.

In particular the above process relates to the process for the preparation of the compound (IIlb) predominantly in its (S)-configuration, this compound also known as S-Metolachlor having herbicidal properties, such process comprising hydrogenation of the imine of the formula (Ib) with hydrogen under elevated pressure in the presence of one or more iridium complexes as catalyst and one or more compounds comprising a carbon-halogen bond as co-catalysts to form the compound of formula (IIb) and subsequent reaction with chloroacetic acid chloride.

Within the group of diphosphine ligands useful according to the present invention are the novel ferrocenyl ligands of formula (VII) or formula (VIII) that have been found to show a significant effect when used as part of iridium complexes as catalysts in a process for the hydrogenation of imines with hydrogen under elevated pressure and in the presence of one or more co-catalysts selected among compounds comprising a carbon-halogen bond as described herein:

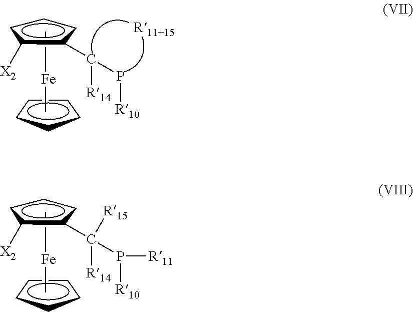

wherein

R′₁₁₊₁₅ represents an C₁-C₈alkylene, alkenylene, or alkynylene bridge optionally with one or more of the carbon atoms substituted with a heteroatom, said bridge optionally being substituted with one or more substituents that may be selected among C₁-C₁₂alkyl, C₃-C₈cycloalkyl, C₆-C₁₂aryl or C₃-C₁₂heteroaryl having heteroatoms preferably selected from the group consisting of O, S and N; the aforementioned groups being unsubstituted or substituted independently of the others by one or more substituents e.g. by C₁-C₆alkyl, C₁-C₆alkoxy, C₃-C₈cycloalkyl, C₃-C₈cycloalkoxy, phenyl, C₁-C₆alkylphenyl, C₁-C₆alkoxy phenyl or C₃-C₈heteroaryl;

X₂ represents a secondary phosphine group;

R′₁₀ and R′₁₁ are each independently of the other C₁-C₁₂alkyl, C₁-C₁₂alkoxy, C₃-C₈cycloalkyl, C₃-C₈cycloalkoxy, C₆-C₁₂aryl, C₃-C₁₂heteroaryl, C₆-C₁₂aryl-C₁-C₁₂alkyl-, C₆-C₁₂aryl-C₁-C₁₂alkoxy- or C₃-C₁₂heteroaryl-C₁-C₁₂alkyl- having heteroatoms preferably selected from the group consisting of O, S and N, the aforementioned groups being unsubstituted or substituted independently of the others by one or more substituents e.g. by C₁-C₆alkyl, C₁-C₆alkoxy, C₃-C₈cycloalkyl, C₃-C₈cycloalkoxy, phenyl, phenyl-C₁-C₆alkyl-(e.g. benzyl), phenyl-C₁-C₆alkoxy-, C₃-C₈heteroaryl, (C₁-C₁₂alkyl)₃Si, (C₆-C₁₂aryl)₃Si, —NH₂, (phenyl)₂N—, (benzyl)₂N—, morpholinyl, piperidinyl, pyrrolidinyl, (C₁-C₁₂alkyl)₂N—, (C₇-C₁₂aralkyl)₂N—, C₁-C₁₂haloalkyl, C₁-C₁₂haloalkoxy, halogen, -ammonium-X₁, —SO₃M₁, —CO₂M₁, —PO₃M₁ or by —COO—C₁-C₆alkyl, wherein M₁ is an alkali metal or hydrogen and X₁ is the anion of a monobasic acid.

R′₁₄ represents hydrogen, OR'₀₀, C₁-C₁alkyl, C₁-C₄fluoroalkyl, C₃-C₈cycloalkyl, C₆-C₁₂aryl or C₃-C₁₂heteroaryl having heteroatoms preferably selected from the group consisting of O, S and N.

R′₁₅ represents an alkyl group, preferably a C₁-C₂₀alkyl group, substituted by at least one group OR′₀₀ or NR′₀₇R′₀₈ and said alkyl group may be further substituted by e.g. one or more substituents such as halogen, C₁-C₆alkyl, C₁-C₆alkoxy, C₃-C₈cycloalkyl, C₃-C₈cycloalkoxy, phenyl, C₁-C₆alkyl phenyl, C₁-C₆alkoxy phenyl, C₃-C₈heteroaryl or halogen. If more than one OR′₀₀ and/or NR′₀₇R′₀₈ group is present then such groups may be identical or different from one another.

R′₀₀ represents hydrogen, H, C₁-C₁₂alkyl, C₁-C₁₂alkenyl, C₁-C₁₂alkynyl, C₃-C₈cycloalkyl, C₆-C₁₂aryl, R′₀₁R′₀₂R′₀₃Si or C₁-C₁₈acyl that is optionally substituted, e.g. by halogen, hydroxy, C₁-C₈alkoxy or R′₀₄R′₀₅N—; R′₀₁, R′₀₂, and R′₀₃ are each, independently of one another, C₁-C₁₂-alkyl, unsubstituted or C₁-C₄alkyl or C₁-C₄alkoxy-substituted C₆-C₁₀aryl or C₇-C₁₂aralkyl; R′₀₄ and R′₀₅ are each, independently of one another, hydrogen, C₁-C₁₂alkyl, C₃-C₈cycloalkyl, C₆-C₁₀aryl or C₇-C₁₂-aralkyl, or R′₀₄ and R′₀₅ together are trimethylene, tetramethylene, pentamethylene or 3-oxapentylene; the aforementioned groups being unsubstituted or substituted independently of the others by one or more substituents e.g. by halogen, C₁-C₆alkyl, C₁-C₆alkoxy, C₃-C₈cycloalkyl, C₃-C₈cycloalkoxy, phenyl, C₁-C₆alkyl phenyl, C₁-C₆alkoxy phenyl, C₃-C₈heteroaryl; R′₀₇ and R′₀₈ independently of one another represents hydrogen, C₁-C₁₂alkyl, C₃-C₈cycloalkyl, C₆-C₁₀aryl or C₇-C₁₂-aralkyl, or R′₀₇ and R′₀₈ together are trimethylene, tetramethylene, pentamethylene or 3-oxapentylene; the aforementioned groups being unsubstituted or substituted independently of the others by one or more substituents e.g. by halogen, C₁-C₆alkyl, C₁-C₆alkoxy, C₃-C₈cycloalkyl, C₃-C₈cycloalkoxy, phenyl, C₁-C₆alkyl phenyl, C₁-C₆alkoxy phenyl, C₃-C₈heteroaryl.

In a preferred embodiment R′₀₀ represents hydrogen, C₁-C₆alkyl or R′₀₁R′₀₂R′₀₃Si.

Preferably R′₁₁₊₁₅ represents an optionally substituted C₃-C₆alkylene bridge and more preferably an optionally substituted C₃-C₅alkylene bridge.

In a preferred embodiment X, represents the group R′₇R′₈P wherein R′₇ and R′₈ independently of the other are as defined for R′₁₀.

Preferably R′₇, R′₈, R′₁₀ and R′₁₁ each independently of the others represent C₁-C₁₂alkyl, C₁-C₁₂alkoxy, C₃-C₈cycloalkyl, C₃-C₈cycloalkoxy, C₆-C₁₂aryl C₃-C₁₂heteroaryl, C₆-C₁₂aryl-C₁-C₁₂alkyl-, C₆-C₁₂aryl-C₁-C₁₂alkoxy- or C₃-C₁₂heteroaryl-C₁-C₁₂alkyl- having heteroatoms preferably selected from the group consisting of O, S and N; the aforementioned groups being unsubstituted or substituted independently of the others by one or more substituents e.g. by one or more groups selected among C₁-C₆alkyl, C₁-C₆alkoxy, C₃-C₈cycloalkyl, C₃-C₈cycloalkoxy, phenyl, phenyl-C₁-C₆alkyl-, phenyl-C₁-C₆alkoxy-, C₃-C₈heteroaryl, (C₁-C₁₂alkyl)₃Si, (C₆-C₁₂aryl)₃Si, —NH₂, (phenyl)₂N—, (benzyl)₂N—, morpholinyl, piperidinyl, pyrrolidinyl, (C₁-C₁₂alkyl)₂N—, (C₇-C₁₂aralkyl)₂N—, C₁-C₁₂haloalkyl, C₁-C₁₂haloalkoxy or halogen; or R′₇ and R′₈ together represents an alkylene bridge having from 2 to 6 carbon atoms that is optionally interrupted by 1 or more —O—, —S— or —N— comprising radicals such as N—H, N—C₁-C₁₂alkyl or N—C₆-C₁₂aryl; and may be unsubstituted or substituted by ═O or by one or more groups of C₁-C₆alkyl, C₁-C₆alkoxy, C₃-C₈cycloalkyl, C₃-C₈cycloalkoxy, phenyl, phenyl-C₁-C₆alkyl-, phenyl-C₁-C₆alkoxy-, or halogen groups; and/or condensed with benzene, pyridine, pyrimidine, furan, thiophene or pyrrole.

R′₇, R^{′}₈, R^{′}₁₀ and R′₁₁ can be substituted in any positions by identical or different radicals, for example by from 1 to 5, preferably from 1 to 3, substituents

More preferably R′₇, R′₈, R′₁₀ and R′₁₁ each independently of the others represent C₁-C₆alkyl; C₄-C₆cycloalkyl that are unsubstituted or have from 1 to 3 C₁-C₄alkyl or C₁-C₄alkoxy substituents; phenyl or benzyl that is unsubstituted or has from 1 to 3 C₁-C₄alkyl, C₁-C₄alkoxy, F, Cl, C₁-C₄fluoroalkyl or C₁-C₄fluoroalkoxy substituents.

R′₁₄ preferably represent hydrogen.

R′₁₅ is preferably a C₁-C₆alkyl group substituted by at least one group OR′₀₀ or NR′₀₇R′₀₈, and more preferably substituted by OR′₀₀, preferably just one OR′₀₀ group, wherein R′₀₀ preferably represents H, C₁-C₄alkyl or R′₀₁R′₀₂R′₀₃Si. Most preferably R′₁₅ is a C₂-C₅alkyl group substituted by one OR′₀₀ group wherein R′₀₀ preferably represents H, C₁-C₃alkyl or R′₀₁R′₀₂R′₀₃Si.

R′₀₁, R′₀₂, R′₀₃ independently of one another preferably represents linear or branched C₁-C₈-alkyl, phenyl or benzyl optionally being substituted by one or more substituents e.g. by one or more C₁-C₄alkyl, C₁-C₄haloalkyl or C₁-C₄alkoxy groups.

The OR′₀₀ group is preferably terminal positioned.

The cyclopentadienyl moiety of the ferrocenyl groups in the above formula (VII) or formula (VIII) may be substituted independently of one another, e.g. by one or more substituents that may the same or different e.g. halogen or a substituent bonded via a C atom, N, atom, S atom, Si atom, a P(O) group or a P(S) group. Such groups may include any of the following that may be further substituted: methyl, ethyl, n- and i-propyl, n-, i- and t-butyl, pentyl, hexyl, cyclohexyl, cyclohexyl methyl, phenyl, benzyl, trimethylsilyl, F, Cl, Br, methylthio, methylsulphonyl, methylsulphoxyl, phenylthio, phenylsulphonyl, phenylsulphoxyl, —CH(O), —C(O)OH, —C(O)—OCH₃, —C(O)—OC₂H₅, —C(O)—NH₂, —C(O)—NHCH₃, —C(O)—N(CH₃)₂, —SO₃H, —S(O)—OCH₃, —S(O)—OC₂H₅, —S(O)₂—OCH₃, —S(O)₂—OC₂H₅, —S(O)—NH₂, —S(O)—NHCH₃, —S(O)—N(CH₃)₂, —S(O)—NH₂, —S(O)₂—NHCH₃, —S(O)₂—N(CH₃)₂, —P(OH)₂, PO(OH)₂, —P(OCH₃)₂, —P(OCH₃)₂, —PO(OCH₃)₂, —PO(OC₂H₅)₂, trifluoromethyl, methylclohexyl, methylcyclohexylmethyl, methylphenyl, dimethylphenyl, methoxyphenyl, dimethoxyphenyl, hydroxymethyl, hydroxyethyl, hydroxypropyl, —CH₂NH₂, —CH₂N(CH₃)₂, —CH₂CH₂NH₂, —CH₂CH₂N(CH₃)₂, methoxymethyl, ethoxymethyl, methoxyethyl, ethoxyethyl, HS—CH₂—, HS—CH₂CH₂—, CH₃S—CH₂—, CH₃S—CH₂CH₂—, —CH₂—C(O)OH, —CH₂CH₂—C(O)OH, —CH₂—C(O)OCH₃, —CH₂CH₂—C(O)OCH₃, —CH₂—C(O)NH₂, —CH₂CH₂—C(O)NH₂, —CH₂—C(O)—N(CH₃)₂, —CH₂—SO₃H, —CH₂CH₂—C(O)N(CH₃)₂, —CH₂CH₂—SO₃H, —CH₂—SO₃CH₃, —CH₂CH₂—SO₃CH₃, —CH₂—SO₂NH₂, —CH₂—SO₂N(CH₃)₂, —CH₂—PO₃H₂, —CH₂CH₂—PO₃H₂, —CH₂—PO(OCH₃), —CH₂CH₂—PO(OCH₃)₂, —C₆H₄—C(O)OH, —C₆H₄—C(O)OCH₃, —C₆H₄—S(O)₂OH, —C₆H₄—S(O)₂OCH₃, —CH₂—O—C(O)CH₃, —CH₂CH₂—O—C(O)CH₃, —CH₂—NH—C(O)CH₃, —CH₂CH₂—NH—C(O)CH₃, —CH₂—O—S(O)₂CH₃, —CH₂CH₂—O—S(O)₂CH₃, —CH₂—NH—S(O)₂CH₃, —CH₂CH₂—NH—S(O)₂CH₃, —P(O)(C₁-C₈alkyl)₂, —P(S)(C₁-C₈alkyl)₂, —P(O)(C₆-C₁₀aryl)₂, —P(S)(C₆-C₁₀aryl)₂, —C(O)—C₁-C₈alkyl and —C(O)—C₆-C₁₀aryl.

The compounds of formula (VII) or formula (VIII) may be present in the form of racemates, mixtures of diastereomers or optically pure stereoisomers. Preferably the compounds are present in enatiomeric excess of the desired isomer, for example, higher than 50%, preferably higher than 60%, more preferably higher than 70% and even more preferably higher than 85%, most preferably the desired isomer is synthesised or isolated in >99% purity relative to any of the other diastereomers having the general formula (VII) or (VIII) respectively.

Compounds of the formula (VII) may be prepared according to the following general reaction scheme:

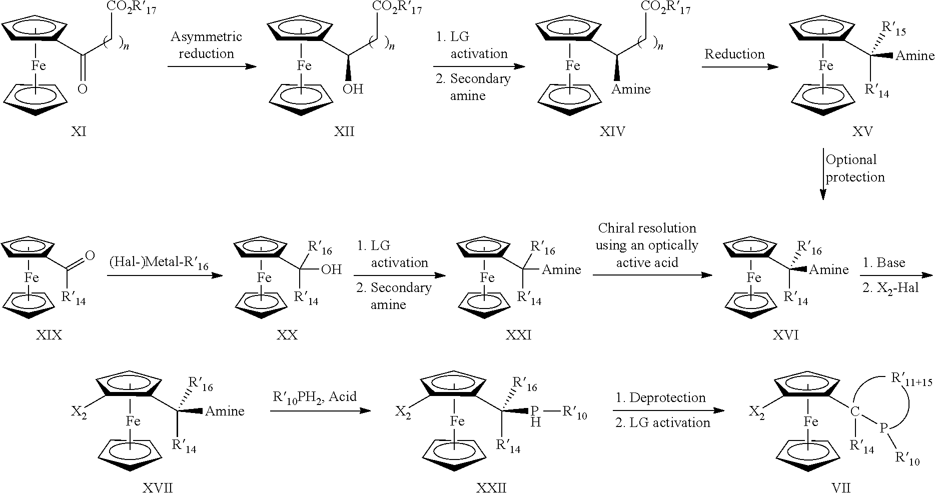

Compounds of the formula (VIII) may be prepared according to the following general reaction scheme:

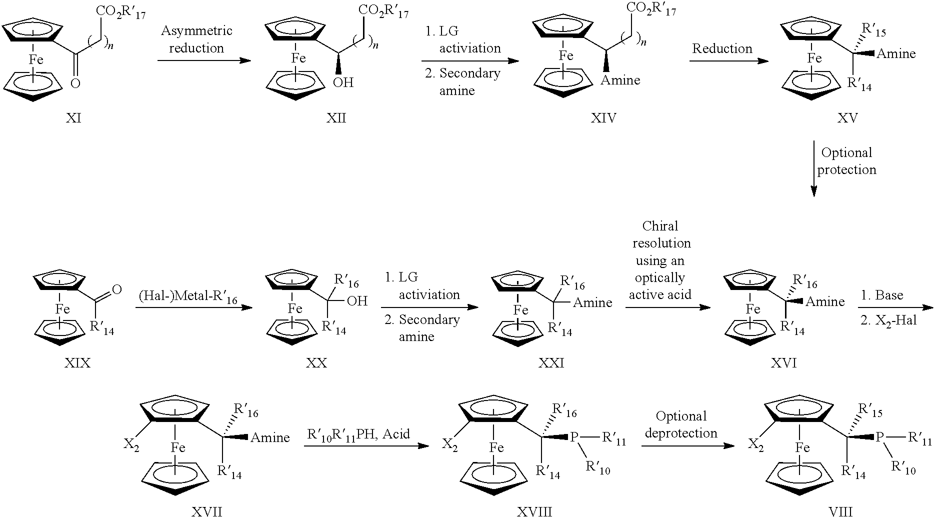

In the above reaction scheme the substituents X₂, R′₁₀, R^{′}₁₁, R^{′}₁₄, R^{′}₁₅ are as defined previously. Hal represents a halogen atom, preferably Br or Cl. Metal is preferably Mg. Amine is a secondary amine, preferably an amine substituent of formula NR′₀₇R′₀₈ wherein R′₀₇ and R′₀₈ are as herein previously defined, and is preferably independently of one another C₁-C₁₂alkyl, C₃-C₈cycloalkyl, C₆-C₁₀aryl or C₇-C₁₂-aralkyl.

When the goal is the preparation of compounds of general formula (VIII) wherein the substituent R′₁₅ comprise at least one hydroxy group (i.e. OR′₀₀ represents OH), it is usually necessary, but dependent on reaction conditions, to protect such group(s) during the course of the above reaction sequence e.g. with a protecting group such as a silyl group of formula R′₀₁R′₀₂R′₀₃Si wherein R′₀₁, R^{′}₀₂, R^{′}₀₃ are as defined previously herein or an acyl group, e.g. C₁-C₁₈acyl that is optionally substituted, e.g. C₁-C₈alkoxycarbonyl groups (for example t-butoxycarbonyl), C₁-C₈alkenyloxycarbonyl groups (for example allyloxycarbonyl), C₆-C₁₂aryl-C₁-C₈alkoxycarbonyl groups (e.g benzoyloxycarbonyl, p-ethoxybenzyloxycarbonyl, o-nitrobenzyloxycarbonyl, p-nitrobenzyloxycarbonyl). Accordingly R^{′}₁₆ is as defined for R′₁₅ but when R′₁₆ comprise a group OR′₀₀, R′₀₀ further includes, besides H, silyl and acyl, other hydroxyl protection groups known in the art such as e.g. C₁-C₈alkyl groups (for example tert-butyl), C₁-C₈alkenyl groups (for example allyl) or C₇-C₁₆aralkyl bonded via an alkyl carbon atom (for example benzyl groups).

R′₁₇ represents an alkyl group, preferably a C₁-C₈alkyl group.

n is an integer having a value of 0 or higher, preferably an integer between 0-11.

Thus, in the above scheme formula (XV) compared with formula (XVI) as well as formula (XVIII) compared with formula (VIII) the difference, respectively, is the optional presence of hydroxy protection groups.

Initially, compounds of formula (XI) are obtained by acylation of ferrocene using known literature procedures (Angew. Chem. Int. Ed. 1971, 10, 191). For simultaneous insertion of a preferred central and planar chirality, the acylated ferrocene (XI) can in principle be converted by any of the methods that are possible to the skilled artisan for this reaction (e.g. J. Am. Chem. Soc. 1957, 79, 2742, J. Organometal. Chem. 1973, 52, 407-424). However, reduction with the so-called CBS reagent (J. Am. Chem. Soc. 1987, 109, 5551-5553, Tetrahdron Lett. 1996, 37, 25-28) is preferable. This procedure ensures that (XII) are produced in very good yields and with very high optical purity. Another conceivable path for preparation of the desired enantiomer-enriched ligands can take place, for example, by preparing the alkylated ferrocenes by means of enantioselective reductive amination. One equally arrives at the enantiomer-enriched ligands with an amine substituent at the stereogenic center in this manner. Other possibilities for introduction of chirality are described in principle in Tetrahedron Asymmetry 1991, 2, 601-612, J. Org. Chem. 1991, 56, 1670-1672, J. Org. Chem. 1994, 59, 7908-7909, J. Chem. Soc., Chem. Commun. 1990, 888-889. The enantiomer-enriched alcohols (XII) that are obtained by the above described CBS reaction can now be converted to derivatives of formula (XIV) in all of the ways that are known to the skilled artisan. Preferably the derivatives are made by replacing the OH function at the stereogenic center by an amino group by forming an intermediate leaving group and subsequently substitution with a secondary amine. Compounds of the general formula (XV) are obtained by reduction of the ester functionality and optionally further elaboration of the hydroxy product to give (XVI). Alternatively (XVI) may be prepared starting from compounds of the general formula (XIX): Addition of an organometallic reagent provides (XX), which may be transformed into the racemic amine adduct (XXI), by a leaving group activation, followed by substitution with a secondary amine. The racemic intermediate may be resolved by using an optically active acid providing (XVI) as an optically enriched product (e.g. J. Am. Chem. Soc. 1970, 92, 5389-5393). Especially preferred is the preparation of dialkylamino derivatives, since these can be employed for the further conversion to (XVII). In this step, the dialkylamino derivatives (XVII) can advantageously be deprotonated in the α position of the cyclopentadienyl ring and then reacted with an electrophile to introduce a phosphine, preferably a diarylphosphine. The deprotonation reaction can take place with all of the agents that are commonly known to the skilled artisan for this purpose, but preferred is the use of the strong bases n-butyllithium (n-BuLi), s-butyllithium (s-BuLi), or t-butyllithium (t-BuLi) in an inert solvent. Preferably the lithium resultingly bonded to ferrocene is converted to a diarylphosphine with a phosphine reagent. Because of the chirality present in the molecule, of the two a positions in the ring, one is preferably deprotonated and substituted (J. Am. Chem. Soc. 1970, 92, 5389-5393). Preferred possibilities as phosphine reagents are compounds that have a leaving group at the phosphorus atom and thus exhibit electrophilic character. Such reagents are sufficiently well known to the skilled artisan (J. Am. Chem. Soc. 1955, 77, 3526-29). The use of diphenylphosphine chloride is preferred. Reaction of (XVII) with a primary phosphine under acidic conditions substitutes the amine of (XVII) with retention of configuration providing (XXII) as an optically enriched product (J. Org. Chem. 1972, 37, 3052-3058. Finally, (VII) may be obtained by a sequential deprotection/leaving group activation procedure facilitating ring closure of the phosphine moiety and R′₁₆. Ferrocenyl ligands with the general formula VIII may also be prepared starting from (XVII). Reaction with a disubstituted phosphine in the presence of an acid displaces the amine of (XVII) with retention of configuration. Finally, an optional deprotection of (XVIII) provides access to (VIII).

The invention further provides complexes of metals selected among the group of transition metals of the Periodic Table with one of the compounds of the formula (VII) or formula (VIII) as ligand. Possible metals are, for example, Cu, Ag, Au, Ni, Co, Rh, Pd, Ir, Ru and Pt. Preferred metals are rhodium and iridium and also ruthenium, platinum, palladium and copper. Particularly preferred metals are ruthenium, rhodium and iridium with iridium being most preferred. The metal complexes can, depending on the oxidation number and coordination number of the metal atom, contain further ligands and/or anions. They can also be cationic metal complexes. Such analogous metal complexes and their preparation have been widely described in the literature. These metal complexes comprising ligands of the formula (VII) or formula (VIII) are preferably homogeneous catalysts or catalyst precursors which can be activated under the reaction conditions which can be used for asymmetric addition reactions onto prochiral, unsaturated, organic compounds. The complexes may also contain further ligands and/or anions. Accordingly the present invention also relates to a process for preparing chiral organic compounds by asymmetric addition of hydrogen onto a carbon-carbon or carbon-heteroatom double bond in prochiral organic compounds, wherein the addition reaction is carried out in the presence of catalytic amounts of at least one metal complex comprising ligands of the formula (VII) or formula (VIII), as well as the use of metal complexes comprising ligands of the formula (VII) or formula (VIII) as homogeneous catalysts for the preparation of chiral organic compounds, preferably for the asymmetric addition of hydrogen onto a carbon-carbon or carbon-heteroatom double bond in prochiral organic compounds.

The novel metal complexes can, for example but preferably, correspond to the general formulae (IVh) or (IVi):

[LᵣMeL₁] (IVh)

[LᵣMeL₁]^{(z+1)}(A⁻)_{z} (IVi)

where L₁ is one of the compounds of the formula (VII) or formula (VIII);

L represents identical or different monodentate, anionic or nonionic ligands, or

L represents identical or different bidentate, anionic or nonionic ligands; r is 2, 3 or 4 when

L is a monodentate ligand or r is 1 or 2 when L is a bidentate ligand; z is I, 2 or 3;

Me is a metal selected from the group consisting of Rh, Ir and Ru; with the metal having the oxidation state 0, 1, 2, 3 or 4;

A⁻ is the anion of an oxy or complex acid as previously described herein;

and the anionic ligands balance the charge of the oxidation state 1, 2, 3 or 4 of the metal.

Monodentate nonionic ligands can, for example, be selected from the group consisting of olefins (for example ethylene, propylene), solvating solvents (nitriles, linear or cyclic ethers, unalkylated or N-alkylated amides and lactams, amines, phosphines, alcohols, carboxylic esters, sulphonic esters), nitrogen monoxide and carbon monoxide:

Suitable polydentate anionic ligands are, for example, allyls (allyl, 2-methallyl), cyclopentadienyl or deprotonated I,3-diketo compounds such as acetylacetonate.

Monodentate anionic ligands can, for example, be selected from the group consisting of halogens (F, Cl, Br, I), pseudohalide (cyanide, cyanate, isocyanate) and anions of carboxylic acids, sulphonic acids and phosphonic acids (carbonate, formate, acetate, propionate, methylsulfonate, trifluoromethylsulphonate, phenylsufonate, tosylate).

Bidentate nonionic ligands can, for example, be selected from the group consisting of linear or cyclic diolefins (for example hexadiene, cyclooctadiene, norbornadiene), dinitriles (malononitrile), unalkylated or N-alkylated carboxylic diamides, diamines, diphosphines, diols, dicarboxylic diesters and disulphonic diesters.

Bidentate anionic ligands can, for example, be selected from the group consisting of anions of dicarboxylic acids, disulphonic acids and diphosphonic acids (for example of oxalic acid, malonic acid, succinic acid, maleic acid, methylenedisulphonic acid and methylenediphosphonic acid).

Preferred metal complexes which are particular suitable for hydrogenations are those where

Me in the above formulae represents iridium or rhodium, and preferably iridium complexes corresponding to those of formula (IV) and formula (IVa) wherein Y corresponds to L₁ as above i.e. ligands of the formula (VII) or formula (VIII).

Metal complexes comprising ligands of the formula (VII) or formula (VIII), by example complexes according to formulae (IVh) or (IVi), are prepared by known methods. Use of such metal complexes as homogenous catalysts for e.g. asymmetric hydrogenation (addition of hydrogen) of prochiral unsaturated organic compounds, e.g. compounds comprising one or more carbon-carbon or carbon-heteroatom double bonds, are generally known from the literature.

The present invention also relates to novel intermediates of formulae (XVI′), (XVII′) and (XXII′), which are precursors to the compounds (VII) and (VIII), and which may by themselves function as ligands useful as part of metal complexes e.g. useful as catalysts in a process for the hydrogenation of imines with hydrogen.

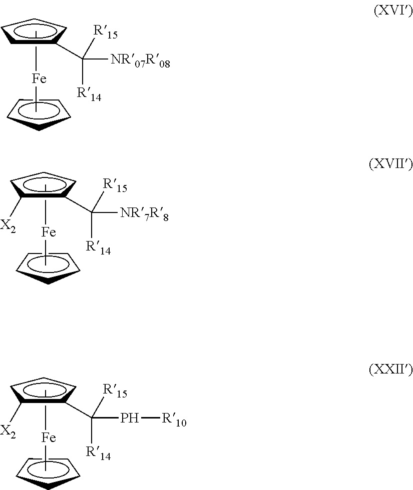

wherein the substituents X₂, R′₀₇, R′₀₈, R′₁₀, R′₁₄, R′₁₅ are as defined previously and wherein the cyclopentadienyl rings in the above structures, independently of one another, may be substituted by one or more substituents, e.g. as described for the compounds of formula (VII) and formula (VIII).

The compounds of formula (XVI), formula (XVII) or formula (XXII) may be present in the form of racemates, mixtures of diastereomers or optically pure stereoisomers. Preferably the compounds are present in enatiomeric excess of the desired isomer, for example, higher than 50%, preferably higher than 60%, more preferably higher than 70% and even more preferably higher than 80%.

The invention is illustrated by the following non-limiting examples:

### Example 1

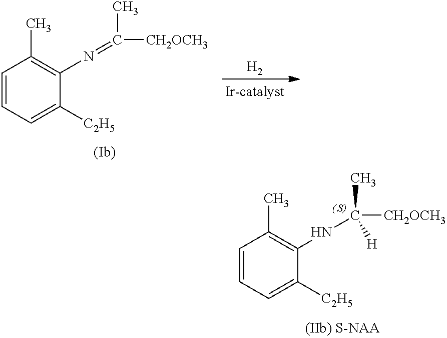

Catalyst preparation: To a flame-dried Schlenk flask was added [Ir(cod)Cl]₂ (1.28 mg, 0.002 mmoles, 0.01 mol %), Xyliphos i.e. the compound {(R)-1-[(S)-2-diphenylphosphino)ferrocenyl]}ethyl-di(3,5-dimethyl-phenyl)phosphine (2.55 mg, 0.004 mmoles, 0.02 mol %) and dry degassed THF (0.5 mL) under argon to give a yellow colored homogenous solution.

To an autoclave was added MEA-imine (Ib) (4.0 g, 19.5 mmoles, 1 eq.) and 2,3,4,5,6-pentafluorobenzyl bromide (7.2 μL, 0.05 mmoles, 0.26 mol %). The reaction vessel was sealed and purged three times with nitrogen under stirring before adding the above catalyst solution through the injector system. The injector system was washed once with dry degassed THF (0.5 mL) before purging three times with hydrogen under stirring. The reaction was initiated by heating to 50° C. and then introducing high pressure hydrogen (30 bar) to the reaction vessel. After 4 hours the reaction vessel was allowed to reach room temperature before releasing the hydrogen. GC yield: >99%. Optical purity: 75 ee (S configuration)—see table 1, entry 19.<

A series of experiments using the above procedure was carried out with various parameters as seen in table 1—entries 1-9 for a comparative purpose.

Results for the screening of aromatic halides and alkyl halides as additives for the catalytic enantioselective hydrogenation of MEA-imine (Ib) to S—NAA (IIb).

TABLE 1

| Entry | Ligand | S/C ratio | Halide | Mol % | Acid | Mol % | Time (h) | Conv. (%) | ee (%) |
|---|---|---|---|---|---|---|---|---|---|
| 1c | Xyliphos | 5000 | — | — | — | — | 4 | 37 | 76 |
| 2c | Xyliphos | 5000 | KCl | 1 | — | — | 4 | 73 | 72 |
| 3c | Xyliphos | 5000 | KCl | 1 | AcOH | 10 | 4 | 3 | 62 |
| 4c | Xyliphos | 5000 | Bu₄NBr | 1 | — | — | 4 | 43 | 74 |
| 5c | Xyliphos | 5000 | Bu₄NBr | 1 | AcOH | 10 | 4 | >99 | 74 |
| 6c | Xyliphos | 5000 | Bu₄NI | 1 | — | — | 4 | 50 | 72 |
| 7c | Xyliphos | 5000 | Bu₄NI | 1 | AcOH | 1 | 4 | 1.2 | Nd |
| 8c | Xyliphos | 5000 | — | | | | | | |
| 9c | Xyliphos | 5000 | — | | AcOH | 10 | | | |
| 10 | Xyliphos | 5000 | PhCl | 0.1 | — | — | 4 | 67 | 73 |
| 11 | Xyliphos | 5000 | 1-Cl-butane | 0.1 | — | — | 10 | 84 | 74 |
| 12 | Xyliphos | 5000 | BnCl | 0.1 | — | — | 10 | 98 | 74 |
| 13 | Xyliphos | 5000 | t-Butylchloride | 0.1 | — | — | 10 | 85 | 74 |
| 14 | Xyliphos | 5000 | iso- | 0.1 | | | 10 | 98 | 74 |
| | | | Butylchloride | | | | | | |
| 15 | Xyliphos | 5000 | PhBr | 0.1 | — | — | 4 | 43 | 76 |
| 16 | Xyliphos | 5000 | PhBr | 0.1 | — | — | 10 | 98 | 74 |
| 17 | Xyliphos | 5000 | 1,2- | 0.1 | | | 10 | 93 | 76 |
| | | | Dibromobutane | | | | | | |
| 18 | Xyliphos | 5000 | 2-Br- | 0.1 | — | — | 4 | >99 | 77 |
| | | | Benzylbromide | | | | | | |
| 19 | Xyliphos | 5000 | PFBnBr | 0.26 | — | — | 4 | >99 | 75 |
| 20 | Xyliphos | 5000 | PFBnBr | 0.26 | AcOH | 10 | 4 | >99 | 74 |
| 21 | Xyliphos | 40000 | PhI | 0.1 | — | — | 4 | 9 | 72 |
| 22 | Xyliphos | 40000 | PFBnBr | 0.26 | — | — | 4 | 84 | 75 |

All experiments are carried out at 50° C. and 30 bar hydrogen pressure. Entries marked with a (c)
are for a comparative purpose
PFBnBr = 2,3,4,5,6-Pentafluorobenzylbromide. AcOH = Acetic acid
S/C = Substrate to catalyst ratio

### Example 2

Examples of how to prepare specific ligands falling within formula (VIII) is provided e.g. via process Route A or Route B, whereas the preparation of a specific ligands falling with formula (VII) by example is provided by example in process Route C.

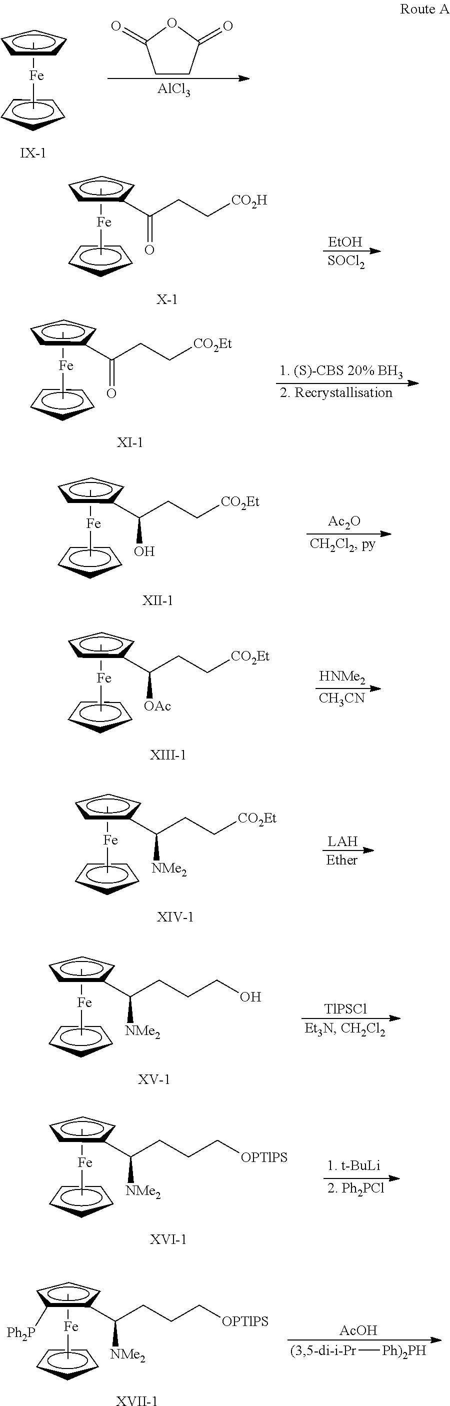

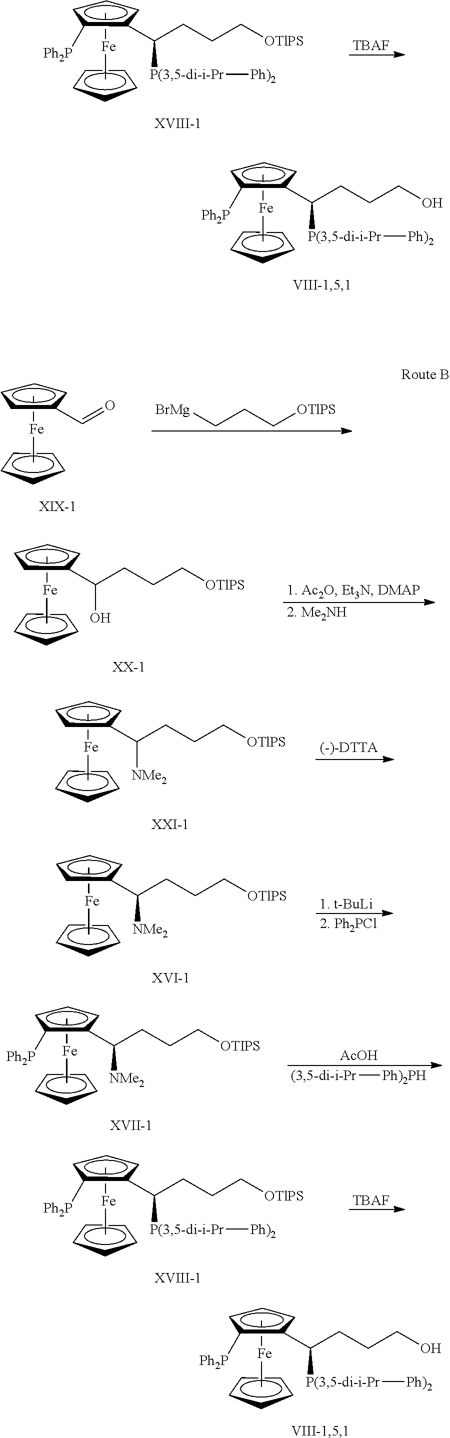

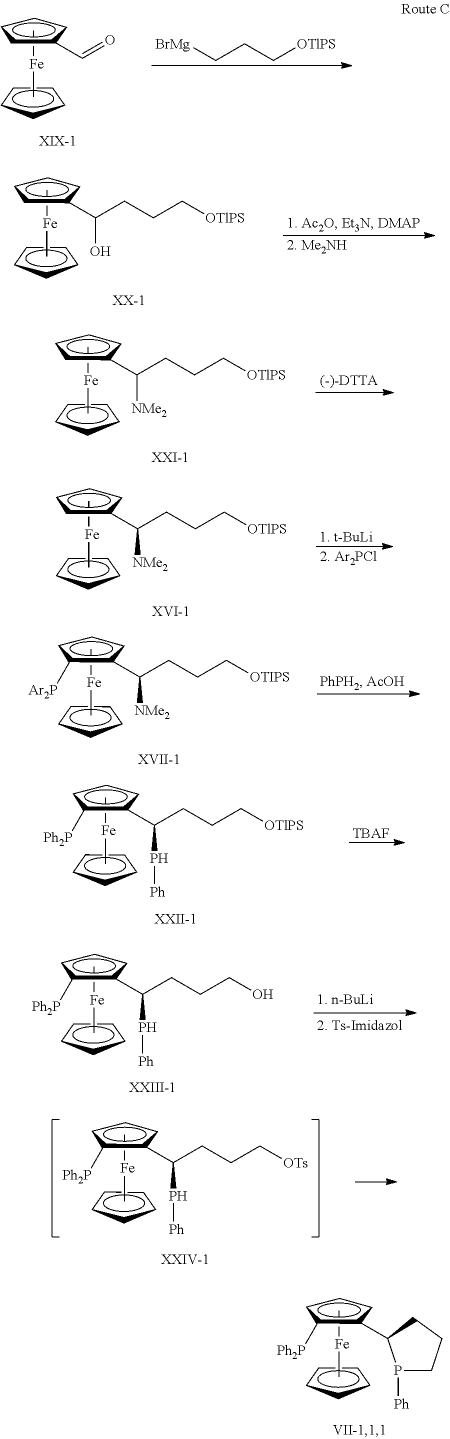

General Procedure for the Synthesis of VIII, Succinic Anhydride Approach (Route A).

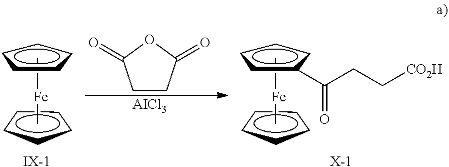

72 g (0,39 mol) of ferrocene and 40 g (0,4 mol) of succinic anhydride were dissolved in DCM (700 ml). The solution was cooled to 0-5° C. using an ice-bath. 70 g (0.53 mol) of aluminum trichloride was added to the solution in small portions. After completed addition, the solution was allowed to warmed up to room temperature and stirred for additional two hours. The reaction was monitored by TLC (EtOAc/PE, 1:5). The mixture was poured into ice-water and stirred for 30 min. The organic phase was separated and the aqueous phase was extracted twice with DCM (200 ml). The combined organic phases were dried over Na₂SO₄ and concentrated in vacuo to yield 80g of crude X-1 which was used in the next step without further purification.

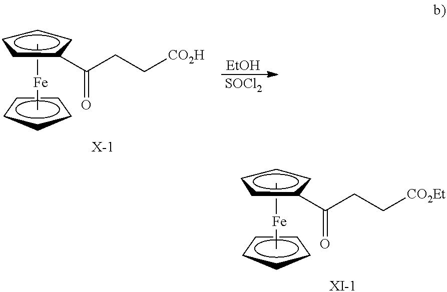

To a 1000 ml three-necked flask, equipped with a reflux condenser and a scrubber for HCl gas adsorption, was added 70 g of crude crude X-1 and ethanol (700 ml). SOCl₂ (40 ml) was slowly added to the solution and the solution was stirred at room temperature for 30 min. The reaction mixture was poured into an ice cooled Na₂CO₃ solution and stirred for 15 min. The aqueous phase was extracted three times with EtOAc (3×200 ml) and the combined organic phases were dried over Na₂SO₄. After filtration, the solution was concentrated to a volume of approximately 100 ml. The crude product solution was kept at room temperature overnight, and upon filtration, XI-1 was isolated as orange crystals. The filtrate was concentrated to dryness and a second crop of XI-1 was obtained by column purification (Eluent: EtOAc/PE, 1:5). ¹H NMR (CDCl₃, 500 MHz): δ 1.29 (t, 2H); 2.69 (t, 2H); 3.07 (t, 2H); 4.20 (q, 2H); 4.26 (s, 5H); 4.33 (s, 2H), 4.80 (s, 2H).

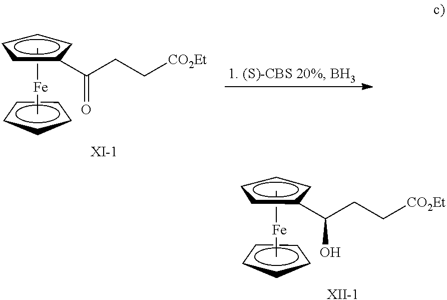

The reaction was conducted under an atmosphere of N₂. (S)-CBS (15 g, 0,054 mol) was dissolved in dry THF (50 ml) and cooled to 0-5° C. using an ice-bath. BH₃ in THF (50 ml, 1M) was added to the solution and stirred for 15 min. XI-1 (128 g, 0,41 mol) was dissolved in dry THF (400 ml) and added into the (S)-CBS solution by cannula. The solution temperature was kept below 5° C. throughout the operation. Another portion of BH₃ dissolved in THF (200 mL, 1M) was added drop wise into the solution via cannula over the course of two hours. Stirring was continued for additional half an hour. The reaction was monitored by TLC (EtOAc/PE, 1:5). The excess BH₃ was quenched by drop wise addition of NH₄Cl (sat. aq. solution) to the reaction mixture. The phases were separated and the aqueous phase was extracted three times with EtOAc. The combined organic phases were dried over Na₂SO₄, and the solvent was removed in vacuo to yield XII-1 (118 g) as a red-orange oil. The optical purity of XII-1 was determined by chiral stationary phase HPLC. Column: AS-H (4.6×250 mm, 5 μm), mobile phase n-Hexane/IPA 20:1, Flow rate 0.8 ml/min, Rtₘᵢₙₒᵣ=17.4 min. Rtₘₐⱼₒᵣ=18.8.

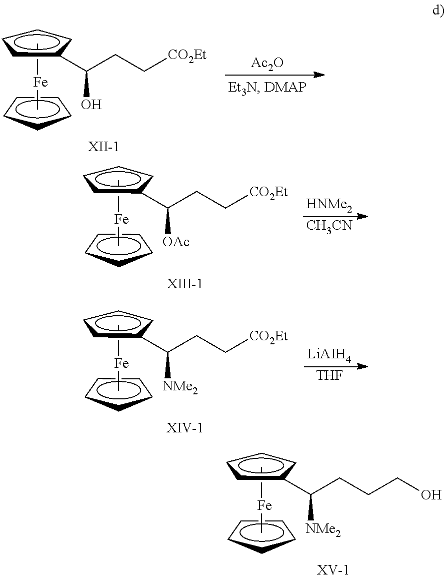

To a solution of XII-1 (100 g) was added dry Et₃N (100 ml) and DMAP (10 g). The reaction was cooled to 10° C. using an ice-water bath followed by the addition of Ac₂O (40 g). After 15 minutes of aging, the reaction was left stirring over night at room temperature under an atmosphere of N₂. Full conversion was checked by TLC (EtOAc/PE=1:5). The solvent was then removed in vacuo and the residue was dissolved in acetonitril (400 ml). 150 mL dimethylamine (150 ml, 33% aq. solution) was added and the mixture was stirred at 45-50° C. for 24 hrs until XIII-1 disappears on TLC (EtOAc/PE=1:5). The solution was poured into water (500 ml) and extracted with EtOAc (3×200 ml). The organic phases were combined and dried over Na₂SO₄. The solvent was removed in vacuo to give a dark red oil. The oil was dried under vacuum overnight and subjected to next step without any further purification. The crude XIV-1 was dissolved in dry THF (400 ml) slowly added drop wise by cannula into a pre-cooled slurry of LiAlH₄ (10 g) and THF (100 ml) at 0° C. The solution was stirred for additional 2 hours and full conversion checked by TLC (EtOAc as eluent). The solution was cooled to −10° C. at which temperature water (10 g) was added slowly drop by drop to quench excess LiAlH₄. The orange slurry was filter and the filtrate was concentrated in vacuo. After drying under vacuum overnight, the red oil had solidified. The solid (XV-1) was washed with hexane and isolated by filtration (42 g). The filtrate was concentrated to dryness and additional product was isolated upon silica column chromatography (EtOAc/PE, 1:1) to yield a red oil (28 g).

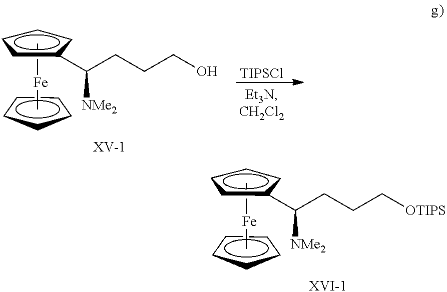

To a solution of XV-1 (26 g) in dry CH₂Cl₂ (100 ml) at 0° C. was added TIPSCl (25 g) followed by Et₃N (15 mL). The reaction was left to warm to rt and stirred over night under an atmosphere of nitrogen. Full conversion was checked by TLC (EtOAc/PE=1:5). The reaction was quenched by the addition of water (50 mL). The organic phase was separated and washed with water (3×100 mL). The organic phase was dried over Na₂SO4 and concentrated in vacuo affording a crude product, which was purified by silica column chromatography (ratio EtOAc/PE=1:4) to yield XVI-1 (34 g) as a red oil.

¹H NMR (CDCl₃, 250 MHz) δ 1.10 (m, 21H), 1.72 (m, 2H), 1.87 (m, 1H), 1.98 (s, 6H), 2.19 (m, 1H), 3.37 (dd, 1H), 3.81 (m, 2H), 4.02 (d, 1H), 4.07 (d, 1H), 4.11 (s, 7H). ¹³C NMR (CDCl₃, 62.5 MHz) δ 12.0 (3C), 18.1 (6C), 27.8, 30.8, 40.4 (2C), 62.8, 63.5, 66.8, 67.1, 67.3, 68.5 (5C), 69.3, 85.5. LC-MS: Highest Mass detected 457. Most abundant mass 412.

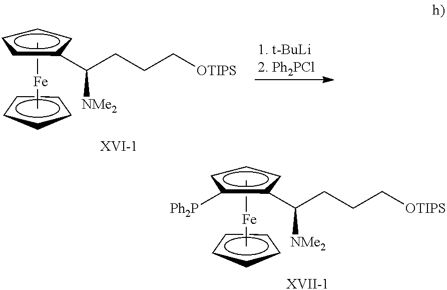

A solution of XVI-1 (10 g) in dry diethylether (50 mL) was cooled to −78° C. A solution of t-BuLi (16 mL, 1.6 M) was added and the reaction was left to warm to room temperature. After 3 hours of aging, the mixture was re-cooled to −78° C. at which temperature Ph₂PCl (6 g) was added. The reaction mixture was allowed to warm to room temperature and stirred for additional 6 hours before slowly poured into an ice-cooled saturated aqueous solution of Na₂CO₃. The mixture was extracted with EtOAc (80 mL) and the combined organic phases were dried over Na)SO₄. The organic solvent was removed in vacuo and the residue was flushed through a silica plug (EtOAc/PE=1:3) to yield XV11-1 a red-orange oil.

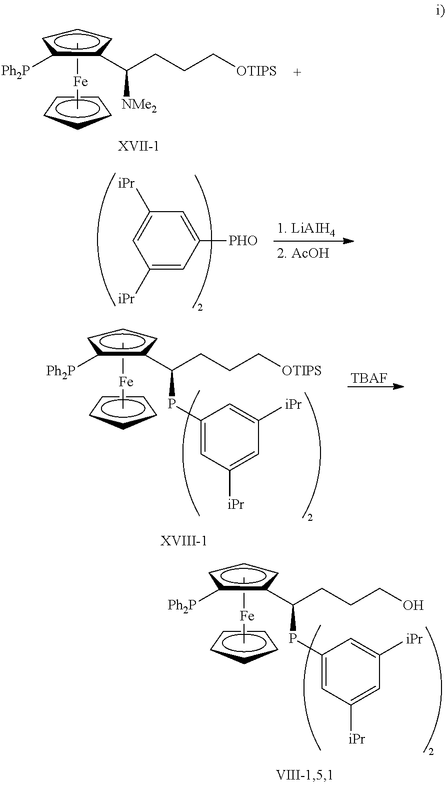

Bis-(3,5-Diisopropyl-phenyl)phosphine oxide (0.7 g) was dissolved in dry THF (5 mL) in a dried Schlenk flask (50 mL volume). The solution was cooled to 0° C., and then LiAlH₄ (0.1 g) was added. The mixture was allowed to stir for 2 hours at 0° C. under an atmosphere of nitrogen. The solvent was removed in vacuo, and the residue was cooled to −78° C. A solution of XVII-1 (1.0 g) in acetic acid (10 mL) was added into the flask. The mixture was heated at 70° C. for 1 hour and 80° C. for 1 hour under N₂ atmosphere. After cooled to room temperature, 30 ml EtOAc and 10 mL distilled water was added and stirred for several minutes. The solution was filtered and, washed with saturated aqueous Na₂CO₃. The organic phase was separated and dried over MgSO₄. The solvent was removed and the residue was flushed through a silica column (EtOAc/PE=1:20) to yield XVIII-1 as a red oil of 10 (0.8 g).

The red oil of XVIII-1 was dissolved in 10 mL of THF. 1 g of TBAF was added. The solution was stirred for 24 hrs at 50° C. under the protection of N,. 20 mL water was added. The solution was extract with EtOAc (3×20mL). The solvent was removed and the residue was flushed through silica column (EtOAc/PE=1:5) to yield 0.6 g of VIII-1,5,1.

General Procedure for the Synthesis of VIII, Ferrocen Carbaldehyde Approach (Route B).

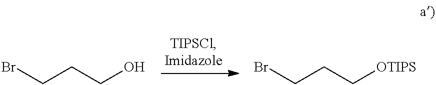

To a 250 ml round-bottomed flask was charged 3-bromo-1-propanole (25.0 g, 180 mmoles, 1.00 eq), Tri-i-propylsilyl chloride (34.7 g, 180 mmoles, 1.00 eq), imidazole (30.6 g, 450 mmoles, 2.50 eq) and dichloromethane (150 mL). The reaction mixture was stirred for 60 hours at rt when it was poured into water (200 mL). The aqeuous phase was extracted with dichloromethane (3×50 mL) and the combined organic phases were dried over magnesium sulfate and concentrated to yield 53.2 g (100.2%) of a colorless oil.

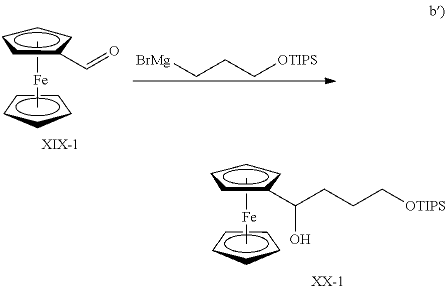

To a flame-dried round-bottomed flask was charged Magnesium (12.15 g, 500 mmoles, 5.00 eq) and dry THF (80 mL) under Argon. The dispersion was heated to reflux and allowed to cool to just under reflux before Dibromoethane (0.1 mL) was added. The mixture was heated until a vigorous reaction started and allowed to react for a few minutes before it was cooled to 0° C. on an ice-bath.

A solution of (3-Bromopropoxy)-tri-i-propylsilane (29.53 g, 100 mmoles, 1.00 eq) in THF (dry, 20 mL) was added to the cooled dispersion of Magnesium over 45 min by a syringe pump. The resulting reaction gave rise to a constant reaction temperature of 9° C. After complete addition the reaction mixture was stirred for another 30 min when full conversion was observed by GC (sample quenched with water). Ferrocenealdehyde (XIX-1) (17.12 g, 80 mmoles, 0.80 eq) in THF (dry, 40 mL) was added over 30 min at 0° C. by a syringe pump. The reaction mixture was stirred for another 15 min before the reaction mixture (except the excess magnesium) was poured into a 1:1 ammoniumchloride (aq, sat)—water mixture (250 mL, 0° C.). The excess magnesium was washed with MTBE and added to the THF-water solution. The resulting mixture was extracted with MTBE (3×100 mL) and the combined organic phases were dried over magnesium sulfate and concentrated to yield 38.9 g (90% (if purity considered to be 80%)) of a dark brown oil (XX-1).

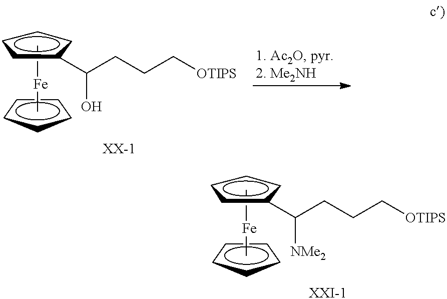

To a round-bottomed flask was charged the produced alcohol from the previous Grignard reaction in step b′) (38.9 g, 80% pure, 72 mmoles), acetic anhydride (30 g, 294 mmoles, 4.0 eq) and pyridine (30 g, 379 mmoles, 5.3 eq). The resulting solution was stirred overnight at rt under Ar when it was poured into water (300 mL) and extracted with MTBE (3×75 mL). The combined organic fractions were concentrated to yield 50 g of a dark oil including residues of acetic acid and pyridine. This material was used without purification in the next step.

To a round-bottomed flask was charged the crude acetylated product (50 g), aqueous dimethylamine (40 mL, 50% solution, ˜400 mmoles) and isopropanol (60 mL). The resulting solution was stirred overnight at rt and further 1 h at 50° C. and 2h at 60° C. before full conversion was observed by TLC. The reaction mixture was poured into water (300 mL) and pH of the aqueous phase measured to be 10. The aqueous solution was extracted with diethylethyer (3×75 mL) and the combined organic phases were dried over magnesiumsulfate and concentrated to yield 37 g of a dark viscous oil (XXI-1).

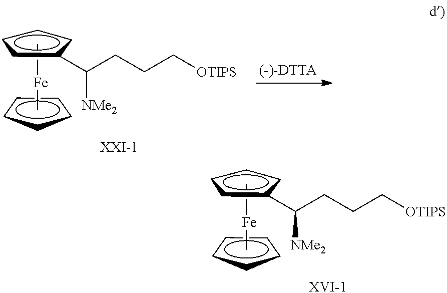

To a solution of amine XXI-1 (1.0 g, 2.2 mmol, 1.0 eq) in isopropyl alcohol (10 mL) was added (2R,3R)-2,3-bis(4-methylbenzoyloxy)succinic acid, hereinafter referred to as (−)-DTTA (844 mg, 2.2 mmol, 1.0 eq). The mixture was heated under stirring to homogeneity and was then allowed to cool to room temperature. The mixture was seeded with seeding crystals (10 mg) and left stoppered at −18° C. overnight. The mixture was decanted and the solid was washed with n-hexane (3×4 mL) and dried to give XVI-1(−)-DTTA (839 mg, 46%) with an enantiomeric excess of 45%. The salt was recrystallised three times in isopropyl alcohol (salt concentration=10% w/v) to provide XVI-1(−)-DTTA (271 mg, 15%) with an enantiomeric excess of 98%.

The optical purity of XVI-1(−)-DTTA was established in the following manner: To a small sample (ca. 20 mg) was added tetrahydrofuran (0.5 mL) and 4 M hydrochloric acid (0.5 mL). The mixture was stirred for 30 min and then basified with concentrated sodium hydroxide (1 mL). The mixture was extracted (n-hexane, 2×1 mL) and the combined organic phase was dried (sodium sulfate), filtered and evaporated to dryness. The resultant oil was dissolved in n-hexane and analysed by chiral stationary phase HPLC (Column: OD-H Chiralcel. Eluent: 0.1% diethylamine, 1% isopropyl alcohol in n-hexane)

The amine XVI-1 was liberated in the following way: To a slurry of XVI-1(−)-DTTA (1.00 g, 1.19 mmol) in 4 M sodium hydroxide (50 mL) was added n-hexane (50 mL). The mixture was stirred for 30 minutes before the phases were separated. The aqueous phase was extracted with hexane (3×30 mL) and the combined organic phase was washed with 4 M sodium hydroxide (20 mL) and water (50 mL) before drying (sodium sulfate), filtration and concentration in vacuo to give amine XVI-1 (476 mg, 84%) as an orange oil.

The compound XVI-1 is converted to a compound falling within general formula (VIII) as provided for under Route A.

### Example 3

Ligands prepared using the synthesis protocols outlined in Route A and B.

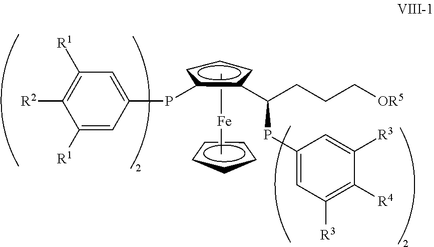

TABLE 2

| | | | | | | ³¹P NMR | | J_{PP} |
|---|---|---|---|---|---|---|---|---|
| Comp. | R¹ | R² | R³ | R⁴ | R⁵ | (δ) ¹ | | (Hz) |
| VIII-1,1,1 | H | H | H | H | H | −25.2 | 2.3 | 13 |
| VIII-1,2,1 | H | H | H | t-Bu | H | −24.8 | 0.2 | 13 |
| VIII-1,3,1 | H | H | H | NMe₂ | H | −24.5 | −2.5 | 6 |
| VIII-1,4,1 | H | H | Me | H | H | −24.3 | 3.2 | 10 |
| VIII-1,5,1 | H | H | i-Pr | H | H | −23.7 | 7.3 | 18 |
| VIII-1,6,1 | H | H | OMe | H | H | −24.8 | 8.8 | 16 |
| VIII-1,7,1 | H | H | Me | OMe | H | −24.2 | 1.4 | 10 |
| VIII-1,8,1 | H | H | Me | NMe₂ | H | −23.6 | 1.5 | 11 |
| VIII-1,9,1 | H | H | Me | NEt₂ | H | −23.6 | 0.7 | 12 |
| VIII-1,10,1 | H | H | Me | N-Pyrrolidyl | H | −24.0 | 2.9 | 15 |
| VIII-1,11,1 | H | H | 1-Naphthyl | | H | −24.5 | −25.4 | 15 |
| VIII-1,8,2 | H | H | Me | NMe₂ | TIPS | −24.0 | 4.5 | — |

^{1 31}PNMR: (CDCl₃, 161 MHz)

¹H and ³¹P NMR data for ligands VIII-1,2,1 and VII-1,1,1

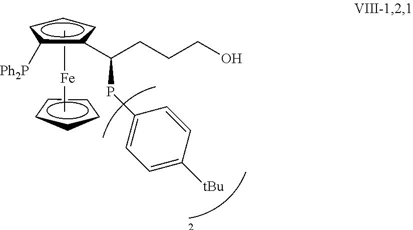

VIII-1,2,1: ¹H NMR (CDCl₃, 500 MHz) δ: 1,27 (s, 9H); 1.30 (s, 9H); 1.44 (m, 1H); 1.60 (m, 1H); 1.91 (m, 1H); 2.34 (m, 1H); 3.31 (m, 1H); 3.44-3.51 (m,2H); 3.89 (s, 5H); 3.98 (s, 1H); 4.27 (m, 1H), 4.41 (s, 1H); 7.14-7.39 (m, 16H); 7.62 (m, 2H).

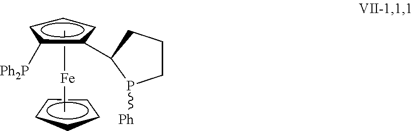

¹H NMR (CDCl₃, 500 MHz). Isomer 1: δ 1.6 (m, 1H), 1.8-2.2 (m, 6H), 2.54 (m, 1H), 3.69 (m, 1H), 3.78 (s, 1H), 4.03 (s, 5H), 4.34 (m, 2H), 7.14-7.17 (m, 10H), 7.39 (m, 3H), 7.59 (m, 2H). Isomer 2: δ 1.6 (m, 1H), 1.89 (m, 2H), 2.05 (m, 1H), 2.23-2.33 (m, 4H); 3.54 (m, 2H), 3.90 (m, 5H), 3.93 (s, 1H), 4.05 (s, 1H), 7.03 (m, 2H), 7.20 (m, 2H), 7.31 (m, 2H), 7.41 (m, 5H), 7.68 (m, 2H).

The two isomers was separated by preparative chiral stationary phase HPLC: CHIRALCEL OD-H column: 0.46 cm I.D.×25 cm L; mobile phase: Hexane/Isopropanol=98 /2; Flow rate: 1.0 ml/min; Wave length: 254 nm; Temp.: 30° C. Isomer 1: (Rentention time, 5.297 min). Isomer 2: (Rentention time, 6.138 min).

### Example 4

Using the procedure outlined in example 1, several experiments were conducted as seen in table 3.

TABLE 3
Results for the screening of aromatic halides and alkyl halides as
additives for the catalytic enantioselective hydrogenation of MEA-
imine (Ib) to S-NAA (IIb). No acid added to the reaction mixture.

| Entry | Ligand | Halide | Mol % | Conv. (%) | Ee (%) |
|---|---|---|---|---|---|
| 1¹ | VII-1,1,1 | BnBr | 0.08 | 97 | 70 |
| 2 | VIII-1,1,1 | PFBnBr | 0.31 | 88 | 73 |
| 3 | | Allylbromide | 0.63 | 71 | 73 |
| 4 | VIII-1,2,1 | PFBnBr | 0.31 | 48 | 78 |
| 5 | | Allylbromide | 0.63 | 57 | 78 |
| 6 | VIII-1,3,1 | PFBnBr | 0.31 | 37 | 74 |
| 7 | | Allylbromide | 0.63 | 34 | 73 |
| 8 | VIII-1,4,1 | PFBnBr | 0.31 | 66 | 75 |
| 9 | | Allylbromide | 0.63 | 41 | 74 |
| 10 | VIII-1,5,1 | PFBnBr | 0.31 | 66 | 76 |
| 11 | | Allylbromide | 0.63 | 56 | 75 |
| 12 | VIII-1,6,1 | PFBnBr | 0.31 | 75 | 71 |
| 13 | | Allylbromide | 0.63 | 70 | 72 |
| 14 | VIII-1,7,1 | PFBnBr | 0.31 | 67 | 75 |
| 15 | | Allylbromide | 0.63 | 62 | 75 |
| 16 | VIII-1,8,1 | PFBnBr | 0.31 | 76 | 79 |
| 17 | | Allylbromide | 0.63 | 66 | 79 |
| 18 | VIII-1,9,1 | PFBnBr | 0.31 | 78 | 79 |
| 19 | | Allylbromide | 0.63 | 58 | 78 |
| 20 | VIII-1,10,1 | PFBnBr | 0.31 | 76 | 80 |
| 21 | | Allylbromide | 0.63 | 67 | 79 |
| 22 | VIII-1,11,1 | PFBnBr | 0.31 | 29 | 13 |
| 23 | | Allylbromide | 0.63 | 20 | 9 |
| 24 | VIII-1,8,2 | PFBnBr | 0.31 | 75 | 80 |
| 25 | VIII-1,8,1 | PFBnBr | 0.15 | 57 | 78 |
| 26 | VIII-1,8,1 | PFBnBr | 0.06 | 46 | 78 |
| 27 | VIII-1,8,1 | none | — | 6 | Nd |
| 28 | VIII-1,8,2 | none | — | 6 | Nd |

General experimental procedure: The experiments were carried out at 50° C., at 30 bar hydrogen pressure, a substrate/catalyst ratio of 40000 and 4 hours reaction time.
¹The experiment was carried out at 50° C., at 80 bar hydrogen pressure, a substrate/catalyst ratio of 100000 and 12 hours reaction time.
PFBnBr = 2,3,4,5,6-Pentafluorobenzylbromide.
Nd = Not determined.

### Example 5

To a flame-dried Schlenk flask was added [Ir(cod)Cl]₂ (3.2 mg, 0.0048 mmoles), ligand VIII-1,8,1 (9.2 mg, 0.0012 mmoles) and dry degassed THF (10.0 mL) under argon to give a yellow colored homogenous solution. To the reaction vessel was added MEA-imine (4.0 g, 19.5 mmoles, 1 eq) and 1,2,3,4,5-pentafluorobenzyl bromide (9.0 μL, 0.059 mmoles, 0.06 mol %). The reaction vessel was sealed and purged three times with nitrogen under stirring before adding 500 μL of the above mentioned catalyst solution through the injector system. The injector system was washed once with dry degassed THF (0.5 mL) before purging three times with hydrogen under stirring. The reaction was initiated by heating to 50° C. and then introducing high pressure hydrogen (30 bar) to the reaction vessel. After 4 hours the reaction vessel was allowed to reach room temperature before releasing the hydrogen.

GC yield>76%, optical purity=79% ee.

### Example 6

To a flame-dried Schlenk flask was added [Ir(cod)Cl]₂ (3.9 mg, 0.0058 mmoles, 0.0005 mol %), ligand VIII-1,8,1 (11.2 mg, 0.015 mmoles, 0.0012 mol %) and dry degassed THF (5.0 mL) under argon to give a yellow colored homogenous solution. To the reaction vessel was added MEA-imine (239.7 g, 1167.6 mmoles, 1 eq) and 1,2,3,4,5-pentafluorobenzyl bromide (106 μL, 0.70 mmoles, 0.06 mol %). The reaction vessel was sealed and purged three times with nitrogen under stirring before adding the catalyst solution through the injector system. The injector system was washed once with dry degassed THF (0.5 mL) before purging three times with hydrogen under stirring. The reaction was initiated by heating to 50° C. and then introducing high pressure hydrogen (80 bar) to the reaction vessel. After 7 hours the reaction vessel was allowed to reach room temperature before releasing the hydrogen.

GC yield>99%, optical purity=79% ee.

### Example 7

To a flame-dried flask was added [Ir(cod)Cl]₂ (0.76 g, 1,1 mmoles, 0.0005 mol %), ligand VIII-1,8,1 (2.12 g, 2.76 mmoles, 0.0012 mol %) and dry degassed THF (817 g) under argon to give a yellow colored homogenous solution. To the autoclave was added MEA-imine (46 Kg, 224.4 moles, 1 eq) and 1,2,3,4,5-pentafluorobenzyl bromide (35.1 g, 0.13 moles, 0.06 mol %). The reaction vessel was purged three times with nitrogen under stirring followed by three times flushing with hydrogen, before adding the catalyst solution through the injector system. The reaction was initiated by introducing high pressure hydrogen (80 bar) to the reaction vessel and heating to 50° C. After 9 hours the reaction vessel was allowed to reach room temperature before releasing the excess hydrogen.

GC yield>99%, optical purity=78% ee.

### Example 8

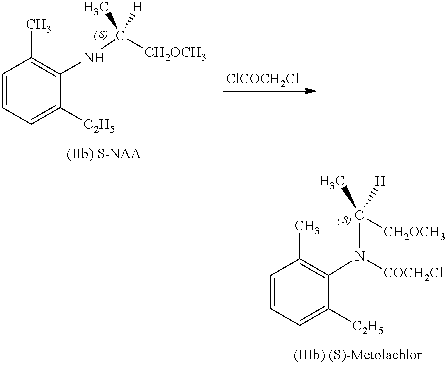

To a 250 mL jacketed reactor equipped with a mechanical stirrer was added S-NAA (IIb) (43.3 g, 0.209 mol) prepared according to the procedure in example 1, hexane (55.1 g) and K₂CO₃ (15 g, 10% aq.) at 25° C. A solution of Chloracetyl chloride (24.8 g, 0.219 mol) in hexane (10.6 g) and NaOH (43.9 g, 20% aq.) were parallel and simultaneously added to the reactor during the course of 30 min, keeping the internal reaction temperature below 30° C. The reaction mixture was stirred for additional 10 min. and the two phases were separated. The organic phase was separated and washed with HCl (50 g, 3.1% aq.), then water (40 g) and subsequently concentration in vacuo to give 56.5 g of product, (S)-Metolachlor.

1. A process for the hydrogenation of an imine with hydrogen under elevated pressure in the presence of an iridium based catalyst, wherein the reaction mixture comprises, in a catalytic effective amount, one or more co-catalysts selected among compounds comprising a carbon-halogen bond.

2. The process according to claim 1 wherein the reaction mixture comprises one or more acids and/or inert solvents.

3. The process according to claim 1, wherein the compounds comprising a carbon-halogen bond is selected among compounds of the formula (VI) 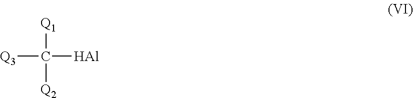
wherein
Hal represents a halogen atom;
Q₁, Q₂, and Q₃ are each, independently of the other, a group selected among H, linear or branched C₁-C₁₂alkyl, C₂-C₁₂alkenyl or C₂-C₁₂alkynyl, C₃-C₈cycloalkyl, heterocycloalkyl bonded via a carbon atom and having from 3 to 8 ring atoms and 1, 2 or 3 hetero atoms from the group O, S and NR₁₉; or is a group selected among C₇-C₁₆aralkyl bonded via an alkyl carbon atom, C₁-C₁₂alkyl substituted by C₃-C₈cycloalkyl, heterocycloalkyl or C₃-C₁₁heteroaryl bonded via a ring carbon atom and having 1, 2 or 3 hetero atoms in the ring selected from the group consisting of O, S and N/NR₁₉; or is a group selected among C₆-C₁₂aryl, or C₃-C₁₁heteroaryl bonded via a ring carbon atom and having 1, 2 or 3 hetero atoms in the ring selected from the group consisting of O, S and N; the aforementioned groups being unsubstituted or substituted by one or more substituents;
or Q₂ and Q₃ together represents a group ═O, ═S, ═NR₁₉, ═CQ₂Q₃;
or Q3 and Q2 form together with Q1 represents a group ≡CO₁;
or Q₁, Q₂, and Q₃ together form, with the carbon atom to which they are bonded, a ring having from 3 to 16 ring carbon atoms, being optionally heterocyclic having from 3 to 16 ring atoms and 1, 2 or 3 hetero atoms from the group O, S and NR₁₉, said ring optionally being substituted with one or more substituents;
R₁₉ represents hydrogen, C₁-C₁₂alkyl, phenyl or benzyl.

4. A The process according to claim 3 wherein Hal represents I, Cl or Br; Q₁ represents linear or branched C₁-C₁₂alkyl or C₂-C₁₂alkenyl, C₆-C₁₂aryl or C₃-C₁₁heteroaryl bonded via a ring carbon atom and having 1, 2 or 3 hetero atoms in the ring, or C₇-C₁₆aralkyl bonded via an alkyl carbon atom; Q₂ represents hydrogen or linear or branched C₁-C₁₂alkyl; Q₃ represents hydrogen or linear or branched C₁-C₁₂alkyl; the aforementioned groups being unsubstituted or substituted independently of the others by one or more substituents;
or Q₂ and Q₃ together represents a group ═O or ═CQ₂Q₃;
or Q₁, Q₂, and Q₃ together form, with the carbon atom to which they are bonded, a benzene ring, being optionally heterocyclic having 1, 2 or 3 hetero atoms, and optionally being substituted with one or more substituents.

5. The process according to claim 1 wherein the co-catalyst is present in amounts of from 0.0001-10 mol %, preferably from 0.0005-5 mol %, more preferably from 0.001-1 mol % and even more preferably from 0.01-1 mol %, based on the imine to be hydrogenated.

6. The process according to claim 1 wherein the iridium complex catalysts is present in amounts of from 0.0001 to 10 mol %, especially from 0.0005 to 10 mol %, and more especially from 0.001 to 5 mol %, based on the imine.

7. The process according to claim 1 wherein the hydrogenation is carried out preferably at a temperature of from −20 to 100° C., especially from 0 to 80° C. and more especially from 10 to 70° C.

8. The process according to claim 1 wherein the hydrogenation is carried out at a hydrogen pressure of 2×10⁵ to 1.5×10⁷ Pa (2 to 150 bar), especially 10⁶ to 10⁷ Pa (10 to 100 bar).

9. The process according to claim 1 where in the imine contains at least one 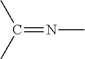
group.

10. The process according to claim 9 wherein the imine is an imine of the formula (I) 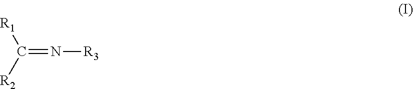
which is hydrogenated to form an amine of formula (II) 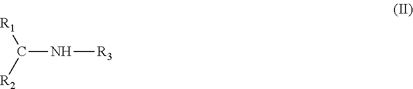
wherein
R₃ is linear or branched C₁-C₁₂alkyl, C₃-C₈cycloalkyl, heterocycloalkyl bonded via a carbon atom and having from 3 to 8 ring atoms and 1 or 2 hetero atoms from the group O, S and NR₆, a C₇-C₁₆aralkyl bonded via an alkyl carbon atom or C₁-C₁₂alkyl substituted by cycloalkyl or heterocycloalkyl or heteroaryl;
or wherein
R₃ is C₆-C₁₂aryl, or C₃-C₁₁heteroaryl bonded via a ring carbon atom and having 1, 2 or 3 hetero atoms from the group O, S and N in the ring; and in either case the aforementioned R₃ groups being unsubstituted or substituted by one or more substituents;
R₁ and R₂ are each independently of the other a hydrogen atom, C₁-C₁₂alkyl or C₃-C₈cycloalkyl, each of which is unsubstituted or substituted independently of the other by one or more substituents; or
R₃ is as defined hereinbefore and R₁ and R₂ together represents an alkylene bridge having from 2 to 6 carbon atoms that is optionally interrupted by 1 or 2 ———, —S— or —NR₆— radicals, and/or unsubstituted or substituted by ═O or as R₁ and R₂ above in the meaning of alkyl, and/or condensed with benzene, pyridine, pyrimidine, furan, thiophene or pyrrole; or
R₂ is as defined hereinbefore and R₁ and R₃ together represents an alkylene bridge having from 2 to 6 carbon atoms that is optionally interrupted by 1 or 2 —O—, —S— or —NR₆— radicals, and/or unsubstituted or substituted by ═O or as R₁ and R₂ above in the meaning of alkyl, and/or condensed with benzene, pyridine, pyrimidine, furan, thiophene or pyrrole.
R₆ represents hydrogen, C₁-C₁₂alkyl, phenyl or benzyl.

11. The process according to claim 10, wherein R₃ is 2,6-di-C₁-C₄alkylphen-1-yl or 2,4-di-C₁-C₄alkylthiophen-3-yl, R₁ is C₁-C₄alkyl, and R₂ is C₁-C₄alkyl, C₁-C₄alkoxymethyl or C ₁-C₄alkoxyethyl.

12. The process according to claim 11, wherein R₃ is 2,6-dimethylphen-1-yl, 2-methyl-6-ethylphen-1-yl or 2,4-dimethylthiophen-3-yl, R₁ is ethyl or methyl, and R₂ is methoxymethyl

13. The process according to claim 12, wherein the imine corresponds to the compound of formula (Ia), (Ib) or (Ic) 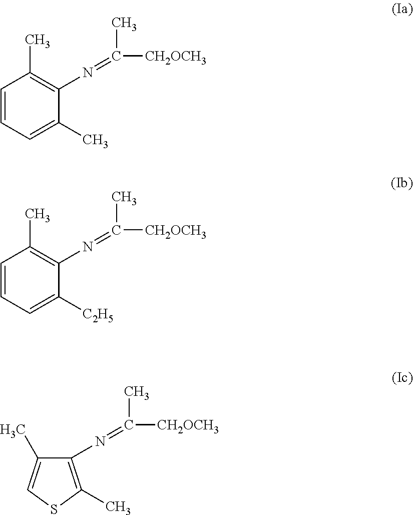
which is hydrogenated to form the amine compound (Ia), (IIb) or (IIc) respectively.

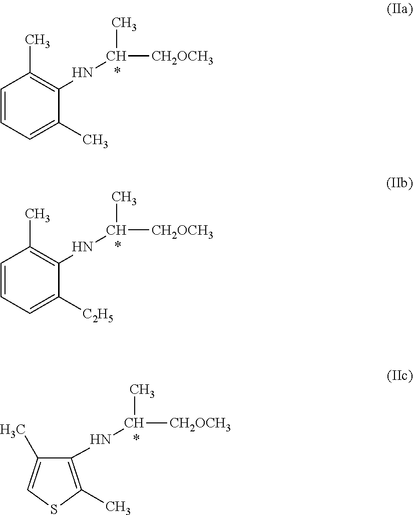

14. A process for the preparation of a compound of formula (IIIa), (IIIb) or (IIIc) 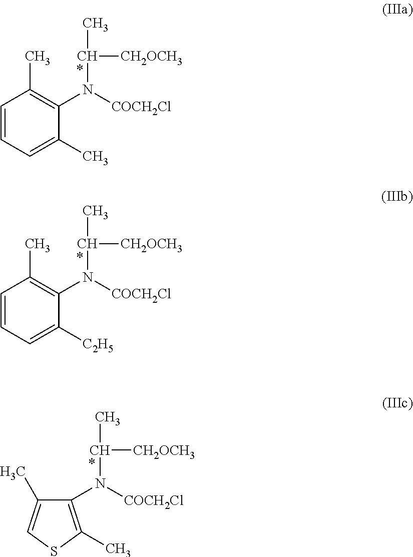
comprising the steps of
i. forming a reaction mixture comprising a) an imine compound of either formula (Ia), (Ib) or (Ic) respectively and optionally an inert solvent, and b) one or more iridium complexes as catalysts and one or more co-catalysts selected among compounds comprising a carbon-halogen bond;
ii. reacting the reaction mixture with hydrogen under elevated preassure to form an amine compound of either formulae (IIa), (IIb) or (IIc) respectively;
iii. reacting the thus formed amine with chloroacetic acid chloride.

15. The process according to claim 14 for the preparation of the compound (IIIb), predominantly in its (S)-configuration.

16. The process according to claim 1 wherein the iridium based catalyst corresponds to the formulae (IV), (IVa), (IVb), (IVc), (IVd), (IVe), (IVf) or (IVg):
[XIrYZ] (IV)
[XIrY]⁺A⁻ (IVa)
[YIrZ₄]⁻M^{+tm (IVb)}
[YIrHZ₂]₂ (IVc)
[YIrZ₃]₂ (IVd)
[YIrZH(A)] (IVe)
[YIrH(A)₂] (IVf)
[YIr(A)₃] (IVg)
wherein X is two olefin ligands or a diene ligand, Y is a ditertiary diphosphine
(a) the phosphine groups of which are bonded to different carbon atoms of a carbon chain having from 2 to 4 carbon atoms, or
(b) the phosphine groups of which are either bonded directly or via a bridge group —CRₐR_{b}— in the ortho positions of a cyclopentadienyl ring or are each bonded to a cyclopentadienyl ring of a ferrocenyl, or
(c) one phosphine group of which is bonded to a carbon chain having 2 or 3 carbon atoms and the other phosphine group of which is bonded to an oxygen atom or a nitrogen atom bonded terminally to that carbon chain, or
(d) the phosphine groups of which are bonded to the two oxygen atoms or nitrogen atoms bonded terminally to a C₂-carbon chain;
with the result that in the cases of (a), (b), (c) and (d) a 5-, 6-, 7-, 8- or 9-membered ring is formed together with the Ir atom;
Z are each independently of the other(s) Cl, Br or I;
A is the anion of an oxy or complex acid;
M⁺ is a cation;
Rₐ and R_{b}, are each independently of the other hydrogen, C₁-C₁₂alkyl, C₁-C₄fluoroalkyl, C₃-C₈cycloalkyl, C₆-C₁₂aryl or C₃-C₁₂heteroaryl having heteroatoms selected from the group consisting of O, S and N, which are unsubstituted or substituted independently by the others by one or more substituents;

17. The process according to claim 16 wherein diphosphine Y correspond to the formula (V), (Va), (Vb), (Vc), (Vd) or (Ve)
R₇R₈P—R₉—PR₁₀R₁₁ (V)
R₇R₈P—O—R₁₂—PR₁₀R₁₁ (Va)
R₇R₈P—NR_{c} R₁₂—PR₁₀R₁₁ (Vb)
R₇R₈P—O—R₁₃—O—PR₁₀R₁₁ (Vc)
R₇R₈P—NR_{c}—R₁₃—NR_{c}—PR₁₀R₁₁ (Vd)
R₇R₈P—NR_{c}—R₉—PR₁₀R₁₁ (Ye)
wherein
R₇, R₈, R₁₀ and R₁₁ each independently of the others represent C₁-C₁₂alkyl, C₁-C₁₂alkoxy, C₃-C₈cycloalkyl, C₃-C₈cycloalkoxy, C₆-C₁₂aryl, C₃-C₁₂heteroaryl, C₆-C₁₂aryl-C₁-C₁₂alkoxy- or C₃-C₁₂heteroaryl-C₁-C ₁₂alkyl- having heteroatoms selected from the group consisting of O, S and N, the aforementioned groups being unsubstituted or substituted independently of the others by one or more substituents; R₇ and R₈ together or R₁₀ and R₁₁ together represents an alkylene bridge having from 2 to 6 carbon atoms that is optionally interrupted by 1 or more —O—, —S— or —NR₆ radicals;
R₉ represents a linear C₂-C₄alkylene that is unsubstituted or substituted by C₁-C₆alkyl, C₃-C₆-cycloalkyl, phenyl, naphthyl or by benzyl; 1,2- or 1,3-cycloalkylene or -cycloalkenylene, -bicycloalkylene or -bicycloalkenylene having from 4 to 10 carbon atoms, the aforementioned groups each being unsubstituted or substituted independently of one another by one or more substituents; 1,2- or 1,3-cycloalkylene or -cycloalkenylene, bicycloalkylene or -bicycloalkenylene having from 4 to 10 carbon atoms, the aforementioned groups each being unsubstituted or substituted independently of one another by one or more substituents; 1,4-butylene substituted in the 2,3-positions by the group 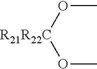
and unsubstituted or substituted in the 1,4-positions by C₁-C₆alkyl, phenyl or by benzyl, wherein R₂₁ and R₂₂ are each independently of the other hydrogen, C₁-C₆alkyl, phenyl or benzyl; 3,4- or 2,4-pyrrolidinylene or 2-methylene-pyrrolidin-4-yl the nitrogen atom of which is substituted by hydrogen, C₁-C₁₂alkyl, phenyl, benzyl, C₁-C ₁₂alkoxycarbonyl, C₁-C₈acyl or by or C₁-C₁₂alkylaminocarbonyl;
1,2-phenylene, 2-benzylene, 1,2-xylylene, 1,8-naphthylene, 2,2’-dinaphthylene or 2,2′-diphenylene, the aforementioned groups each being unsubstituted or substituted independently of one another by one or more substituents; or
R₉ represents an optionally substituted ferrocenyl radical;
R₁₂ is linear C₂- or C₃-alkylene that is unsubstituted or substituted; 1,2- or
1,3-cycloalkylene or -cycloalkenylene, -bicycloalkylene or -bicycloalkenylene having from 4 to 10 carbon atoms, the aforementioned groups each being unsubstituted or substituted independently of the others by one or more groups; 3,4- or 2,4-pyrrolidinylene or 3-methylene-pyrrolidin-4-yl the nitrogen atom of which is substituted by hydrogen, C₁-C₁₂alkyl, phenyl, benzyl, C₁-C₁₂alkoxycarbonyl, C₁-C₈acyl or by C₁-C₁₂alkylaminocarbonyl; 1,2-phenylene, 2-benzylene, 1,2-, 2,3- or 1,8-naphthylene, the aforementioned groups each being unsubstituted or substituted independently of the others by one or more groups R₁₃ is linear C₂alkylene that is unsubstituted or substituted;
1,2-cycloalkylene or -cycloalkenylene, -bicycloalkylene or -bicycloalkenylene having from 4 to 10 carbon atoms, each of which is unsubstituted or substituted by one or more groups; 3,4-pyrrolidinylene the nitrogen atom of which is substituted by hydrogen, phenyl, benzyl, C₁-C₁₂alkoxycarbonyl or by C₁-C₁₂alkylaminocarbonyl; 1,2-phenylene that is unsubstituted or substituted by C₁-C₆alkyl, or is a radical, less two hydroxy groups in the ortho positions, of a mono- or di-saccharide;
R_{c} is hydrogen, C₁-C₆alkyl, phenyl or benzyl.

18. The process according to claim 17 wherein R₉ is represented by the formulae 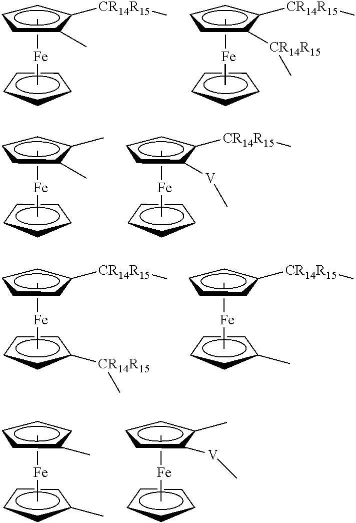
wherein
R₁₄ and R₁₅ independently of one another, each represent hydrogen, C₁-C₂₀alkyl, C₁-C₄fluoroalkyl, C₃-C₈cycloalkyl, C₆-C₁₂aryl or C₃-C₁₂heteroaryl having heteroatoms selected from the group consisting of O, S and N, the aforementioned groups being unsubstituted or substituted independently of the others by one or more substituents; or one of R₁₄ or R₁₅ is connected through a bridging group with the adjacent phosphor atom in the secondary phosphine group of which the carbon atom bearing R₁₄ and R₁₅ is attached to; and wherein the cyclopentadienyl rings, independently of one another, may be substituted by one or more substituents;
V represents an optionally substituted C₆-C₂₀ arylene or C₃-C₁₆ heteroarylene group;

19. The process according to claim 16 wherein Y contains at least one chiral carbon atom and X represents an C₂-C₁₂alkylene olefin ligand or a diene ligand selected among open-chain or cyclic dienes having from 4 to 12 carbon atoms.

20. The process according to claim 1, wherein the co-catalyst is present in an amount that enhances the reaction rate and/or the turnover number of the iridium based catalyst.

21. The process according to claim 1 wherein the use of one or more compounds selected among phosphonium-, metal- and/or ammonium-halides is excluded.

22. The process according to claim 1 wherein the use of an acid is excluded.

23. Compounds of the formula (VII) or formula (VIII) in the form of racemates, mixtures of stereoisomers or optically pure stereoisomers 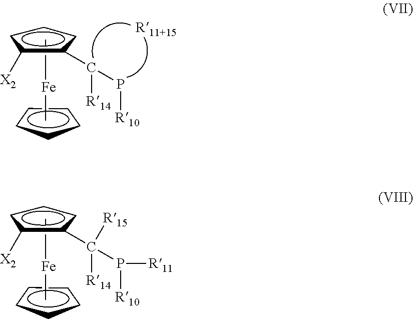
wherein
R′₁₁₊₁₅ represents an C₁-C₈alkylene, alkenylene, or alkynylene bridge optionally with one or more of the carbon atoms substituted with a heteroatom selected from the group consisting of O, S and N, said bridge optionally being substituted with one or more substituents;
X₂ represents a secondary phosphine group;
R′₁₀ and R′₁₁ are each independently of the other represents C₁-C₁₂alkyl, C₁-C₁₂alkoxy, C₃-C₈cycloalkyl, C₃-C₈cycloalkoxy, C₆-C₁₂aryl, C₃-C₁₂heteroaryl, C₆-C₁₂aryl-C₁-C₁₂alkoxy- or C₃-C₁₂heteroaryl-C₁-C₁₂alkyl- having heteroatoms selected from the group consisting of O, S and N, the aforementioned groups being unsubstituted or substituted by one ore more substituents;
R′₁₄ represents hydrogen, OR′₀₀, C₁-C₁₂alkyl, C₁-C₄fluoroalkyl, C₃-C₈cycloalkyl, C₆-C₁₂aryl or C₃-C₁₂heteroaryl having heteroatoms selected from the group consisting of O, S and N, the aforementioned groups being unsubstituted or substituted by one ore more substituents;
R′₁₅ represents C₁-C₂₀alkyl substituted by at least one group OR′ ₀₀ or NR′₀₇R′₀₈ and said alkyl group may be further substituted R′₀₀ represents hydrogen, H, C₁-C₁₂alkenyl, C₁-C₁₂alkynyl, C₃-C₈cycloalkyl, C₆-C₁₂aryl, R′₀₁R′₀₂R′₀₃Si, or C₁-C₁₈acyl that is optionally substituted;
R′₀₇ and R′₀₈ independently of one another represents hydrogen, C₁-C₁₂alkyl, C₃-C₈cycloalkyl, C₆-C₁₀aryl or C₇-C₁₂-aralkyl, or R′₀₇ and R′₀₈ together are trimethylene, tetramethylene, pentamethylene or 3-oxapentylene; the aforementioned groups optionally being substituted independently of the others;
and
the cyclopentadienyl rings, independently of one another, may be substituted by one or more substituents.

24. The compounds according to claim 23 wherein X₂ represents the group R′₇R′₈P wherein R′₇ and R′₈ independently of the other are as defined for R′₁₀.

25. Complexes of metals selected from the group of transition metals of the Periodic Table with compounds of the formula (VII) or formula (VIII) as defined in claim 23 as ligands.

26. The metal complexes according to claim 25 which correspond to the formulae (IVh) and (IVi)
[LᵣMeL₁] (IVh)
[LᵣMeL₁]^{(z+)}(A⁻)_{z} (IVi)
where L₁ is one of the compounds of the formula (VII) or formula (VIII);
L represents identical or different monodentate, anionic or nonionic ligands, or L represents identical or different bidentate, anionic or nonionic ligands; r is 2, 3 or 4 when L is a monodentate ligand or r is 1 or 2 when L is a bidentate ligand; z is 1,2 or 3;
Me is a metal selected from the group consisting of Rh, Ir and Ru; with the metal having the oxidation state 0, 1, 2, 3 or 4;
A⁻ is the anion of an oxy or complex acid;
and the anionic ligands balance the charge of the oxidation state 1, 2, 3 or 4 of the metal.

27. A process for preparing chiral organic compounds by asymmetric addition of hydrogen onto a carbon-carbon or carbon-heteroatom double bond in prochiral organic compounds, wherein the addition reaction is carried out in the presence of catalytic amounts of at least one metal complex according to claim 25.

28. The use of metal complexes according to claim 25 as homogeneous catalysts for the preparation of chiral organic compounds, preferably for the asymmetric addition of hydrogen onto a carbon-carbon or carbon-heteroatom double bond in prochiral organic compounds.

29. The use of the metal complexess according to claim 25 in a process according to claim 1.

30. Compounds of the formula (XVI′) 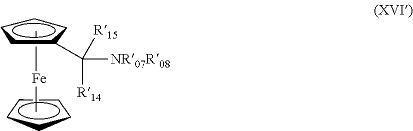
wherein the substituents X₂, R′₀₇, R′₀₈, R′₁₀, R′₁₄, R′₁₅ are as defined in claim 23 and wherein the cyclopentadienyl rings, independently of one another, may be substituted by one or more substituents.

31. Compounds of the formula (XVII′) 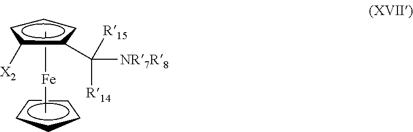
wherein the substituents X₂, R′₀₇, R′₀₈, R′₁₀, R′₁₄, R′₁₅ are as defined in claim 23 and wherein the cyclopentadienyl rings, independently of one another, may be substituted by one or more substituents.

32. Compounds of the formula (XXII′) 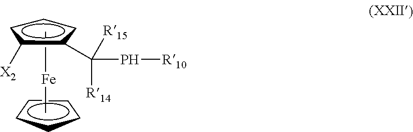
wherein the substituents X₂, R′₀₇, R′₀₈, R′₁₀, R′₁₄, R′₁₅ are as defined in claim 23 and wherein the cyclopentadienyl rings, independently of one another, may be substituted by one or more substituents

33. A process for hydrogenating a prochiral ketimine in the presence of an effective amount of at least one chiral iridium catalyst and at least one co-catalysts comprising a carbon-halogen bond, the co-catalyst being present in an amount such that a hydrogenation reaction rate and/or turnover number of the chiral iridium catalyst is increased by 10% or more compared to a similar reaction under same conditions but without the co-catalyst being present, to produce an optical isomer of an amine having an enantiomeric excess higher than 50%, preferably higher than 70% and even more preferably higher than 80%.
